SEQUENCE LISTING (8 sequences)

SEQ ID NO: 1 - DNA, 2451 bp, Homo sapiens
agaggagtgtttagctccttcccttactctaccttgctcctacttttctctaagtcaacatgagtcgacagtttagttccaggtctgggtaccgaagtggagggggcttcagctctggctctgctgggatcatcaactaccagcgcaggaccaccagcagctccacacgccgcagtggaggaggtggtgggagattttcaagctgtggtggtggtggtggtagctttggtgctggtggtggatttggaagtcggagtcttgttaaccttggtggcagtaaaagcatctccataagtgtggctagaggaggtggacgtggtagtggctttggtggtggttatggtggtggtggctttggtggtggtggctttggtggtggtggctttggtggaggtggcattgggggtggtggctttggtggttttggcagtggtggtggtggttttggtggaggtggctttgggggtggtggatatgggggtggttatggtcctgtctgccctcctggtggcatacaagaagtcactatcaaccagagccttcttcagcccctcaatgtggagattgaccctgagatccaaaaggtgaagtctcgagaaagggagcaaatcaagtcactcaacaaccaatttgcctccttcattgacaaggtgaggttcctggagcagcagaaccaggtactgcaaacaaaatgggagctgctgcagcaggtagatacctccactagaacccataatttagagccctactttgagtcattcatcaacaatctccgaaggagagtggaccaactgaagagtgatcaatctcggttggattcggaactgaagaacatgcaggacatggtggaggattaccggaacaagtatgaggatgaaatcaacaagcggacaaatgcagagaatgaatttgtgaccatcaagaaggatgtggatggtgcttatatgaccaaggtggaccttcaggccaaacttgacaacttgcagcaggaaattgatttccttacagcactctaccaagcagagttgtctcagatgcagactcaaatcagtgaaactaatgtcatcctctctatggacaacaaccgcagtctcgacctggacagcatcattgctgaggtcaaggcccagtacgaggatatagcccagaagagcaaagctgaggccgagtccttgtaccagagcaagtatgaagagctgcagatcactgctggcagacatggggatagtgtgagaaattcaaagatagaaatttctgagctgaatcgtgtgatccagagacttagatctgaaatcgacaatgtcaagaagcagatctccaacttgcagcagtccatcagtgatgcagagcagcgtggcgagaatgccctcaaggatgccaagaacaagctgaatgacctggaggatgccctgcagcaggccaaggaagacctggcccgcctgctgcgcgactaccaggagctgatgaacaccaagctggccctggatctggagattgccacctacaggaccctcctggagggagaagaaagcaggatgtctggagaatgtgccccgaacgtgagtgtgtctgtgagcacaagccacaccaccatcagtggaggtggcagccgaggaggtggcggcggtggctacggctctggaggtagcagctatggctccggaggtggtagctatggttctggaggtggcggcggcggcggccgtggcagctatggctccggaggtagcagctacggctccggaggtggcagctatggctctggaggtggcggcggcggccatggcagctacggctccggaagcagcagtgggggctacagaggtggctctggaggcggcggcggcggcagctctggcggccggggctctggcggcgggagctctggaggctccataggaggccggggatccagctctgggggtgtcaagtcctctggtggcagttccagcgtgaagtttgtttctaccacttattccggagtaaccagataaagagatgccctctgtttcattagctctagttctcccccagcatcactaacaaatatgcttggcaagaccgaggtcgatttgtcccagccttaccggagaaaagagctatggttagttacactagctcatcctattcccccagctctttcttttctgctgtttcccaatgaagttttcagatcagtggcaatctcagtcccctggctatgaccctgctttgttctttccctgagaaacagttcagcagtgaccaccacccacatgacatttcaaagcacctccttaagccagccagagtaggaccagttagacccagggtgtggacagctccttagcatcttatctctgtgctgttttggttttgtacataaggtgtaagcaagttgtttttcttttgtggagaggtcttaaactccccatttccttgttttgctgcaataaactgcatttgaaattc

SEQ ID NO: 2 - DNA, 2320 bp, Homo sapiens
tcgacagctctctcgcccagcccagttctggaagggataaaaagggggcatcaccgttcctgggtaacagagccaccttctgcgtcctgctgagctctgttctctccagcacctcccaacccactagtgcctggttctcttgctccaccaggaacaagccaccatgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcactagtggtggcagcttcaggaaccggtttggtgctggtgctggaggcggctatggctttggaggtggtgccggtagtggatttggtttcggcggtggagctggtggtggctttgggctcggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagactgtgaggcagaacctggagccgttgttcgagcagtacatcaacaacctcaggaggcagctggacagcatcgtgggggaacggggccgcctggactcagagctgagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaagtatgaggagctgcagcagacagctggccggcatggcgatgacctccgcaacaccaagcatgagatctctgagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaagaacctgctgcaagtcactgccttccaagtgcagcaacccagcccatggagattgcctcttctaggcagttgctcaagccatgttttatccttttctggagagtagtctagaccaagccaattgcagaaccacattctttggttcccaggagagccccattcccagcccctggtctcccgtgccgcagttctatattctgcttcaaatcagccttcaggtttcccacagcatggcccctgctgacacgagaacccaaagttttcccaaatctaaatcatcaaaacagaatccccaccccaatcccaaattttgttttggttctaactacctccagaatgtgttcaataaaatgcttttataatataaaaaaaaaaaaaaaaaaa

SEQ ID NO: 3 - DNA, 2162 bp, Homo sapiens
cactccctgggctaaacagcatcaccatgtctgttcgatacagctcaagcaagcactactcttcctcccgcagtggaggaggaggaggaggaggaggatgtggaggaggaggaggagtgtcatccctaagaatttctagcagcaaaggctcccttggtggaggatttagctcaggggggttcagtggtggctcttttagccgtgggagctctggtgggggctgctttgggggctcatcaggtggctatggaggattaggaggttttggtggaggtagctttcgtggaagctatggaagtagcagctttggtgggagttatggaggcagctttggagggggcagtttcggaggtggcagctttggtgggggcagctttggtggaggcggctttggtggaggcggctttggaggaggctttggtggtggatttggaggagatggtggccttctctctggaaatgaaaaagtaaccatgcagaatctgaatgaccgcctggcttcctacttggacaaagttcgggctctggaagaatcaaactatgagctggaaggcaaaatcaaggagtggtatgaaaagcatggcaactcacatcagggggagcctcgtgactacagcaaatactacaaaaccatcgatgaccttaaaaatcagattctcaacctaacaactgataatgccaacatcctgcttcagatcgacaatgccaggctggcagctgatgacttcaggctgaagtatgagaatgaggtagctctgcgccagagcgtggaggctgacatcaacggcctgcgtagggtgctggatgagctgaccctgaccaaggctgacctggagatgcaaattgagagcctgactgaagagctggcctatctgaagaagaaccacgaggaggaaatgaaagaccttcgaaatgtgtccactggtgatgtgaatgtggaaatgaatgctgccccgggtgttgatctgactcaacttctgaataacatgagaagccaatatgaacaacttgctgaacaaaaccgcaaagatgctgaagcctggttcaatgaaaagagcaaggaactgactacagaaattgataataacattgaacagatatccagctataaatctgagattactgaattgagacgtaatgtacaagctctggagatagaactacagtcccaactggccttgaaacaatccctggaagcctccttggcagaaacagaaggtcgctactgtgtgcagctctcacagattcaggcccagatatccgctctggaagaacagttgcaacagattcgagctgaaaccgagtgccagaatactgaataccaacaactcctggatattaagatccgactggagaatgaaattcaaacctaccgcagcctgctagaaggagagggaagttccggaggcggcggacgcggcggcggaagtttcggcggcggctacggcggcggaagctccggcggcggaagctccggcggcggccacggcggcggccacggcggcagttccggcggcggctacggaggcggaagctccggcggcggaagctccggcggcggctacgggggcggaagctccagcggcggccacggcggcagttccagcggcggctacggtggtggcagttccggcggcggcggcggcggctacgggggcggcagctccggcggcggcagcagctccggcggcggatacggcggcggcagctccagcggaggccacaagtcctcctcttccgggtccgtgggcgagtcttcatctaagggaccaagatactaacaaaaccagagtaatcaagacaattattgaagaggtggcgcccgacggtagagttctttcatctatggttgaatcagaaaccaagaaacactactattaaactgcatcaagaggaaagagtctcccttcacacagaccattatttacagatgcatggaaaacaaagtctccaagaaaacacttctgtcttgatggtctatggaaatagaccttgaaaataaggtgtctacaaggtgttttgtggtttctgtatttcttcttttcactttaccagaaagtgttctttaatggaaagaaaaacaactttctgttctcatttactaatgaatttcaataaactttcttactgatgcaaactaaaaaaaaaaaaaaaaaaaaaaa

SEQ ID NO: 4 - DNA, 1653 bp, Homo sapiens
acccgagcaccttctcttcactcagccaactgctcgctcgctcacctccctcctctgcaccatgaccacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctacgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacacagatcccactggaagatcccctctcctgcccaagcacttcacagctggaccctgcttcaccctcaccccctcctggcaatcaatacagcttcattatctgagttgcataaaaaaaaaaaaaaaaaaa

SEQ ID NO: 5 - protein, 644 aa, Homo sapiens
MSRQFSSRSGYRSGGGFSSGSAGIINYQRRTTSSSTRRSGGGGGRFSSCGGGGGSFGAGGGFGSRSLVNLGGSKSISISVARGGGRGSGFGGGYGGGGFGGGGFGGGGFGGGGIGGGGFGGFGSGGGGFGGGGFGGGGYGGGYGPVCPPGGIQEVTINQSLLQPLNVEIDPEIQKVKSREREQIKSLNNQFASFIDKVRFLEQQNQVLQTKWELLQQVDTSTRTHNLEPYFESFINNLRRRVDQLKSDQSRLDSELKNMQDMVEDYRNKYEDEINKRTNAENEFVTIKKDVDGAYMTKVDLQAKLDNLQQEIDFLTALYQAELSQMQTQISETNVILSMDNNRSLDLDSIIAEVKAQYEDIAQKSKAEAESLYQSKYEELQITAGRHGDSVRNSKIEISELNRVIQRLRSEIDNVKKQISNLQQSISDAEQRGENALKDAKNKLNDLEDALQQAKEDLARLLRDYQELMNTKLALDLEIATYRTLLEGEESRMSGECAPNVSVSVSTSHTTISGGGSRGGGGGGYGSGGSSYGSGGGSYGSGGGGGGGRGSYGSGGSSYGSGGGSYGSGGGGGGHGSYGSGSSSGGYRGGSGGGGGGSSGGRGSGGGSSGGSIGGRGSSSGGVKSSGGSSSVKFVSTTYSGVTR

SEQ ID NO: 6 - protein, 590 aa, Homo sapiens
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 7 - protein, 584 aa, Homo sapiens
MSVRYSSSKHYSSSRSGGGGGGGGCGGGGGVSSLRISSSKGSLGGGFSSGGFSGGSFSRGSSGGGCFGGSSGGYGGLGGFGGGSFRGSYGSSSFGGSYGGSFGGGSFGGGSFGGGSFGGGGFGGGGFGGGFGGGFGGDGGLLSGNEKVTMQNLNDRLASYLDKVRALEESNYELEGKIKEWYEKHGNSHQGEPRDYSKYYKTIDDLKNQILNLTTDNANILLQIDNARLAADDFRLKYENEVALRQSVEADINGLRRVLDELTLTKADLEMQIESLTEELAYLKKNHEEEMKDLRNVSTGDVNVEMNAAPGVDLTQLLNNMRSQYEQLAEQNRKDAEAWFNEKSKELTTEIDNNIEQISSYKSEITELRRNVQALEIELQSQLALKQSLEASLAETEGRYCVQLSQIQAQISALEEQLQQIRAETECQNTEYQQLLDIKIRLENEIQTYRSLLEGEGSSGGGGRGGGSFGGGYGGGSSGGGSSGGGHGGGHGGSSGGGYGGGSSGGGSSGGGYGGGSSSGGHGGSSSGGYGGGSSGGGGGGYGGGSSGGGSSSGGGYGGGSSSGGHKSSSSGSVGESSSKGPRY

SEQ ID NO: 8 - protein, 472 aa, Homo sapiens
MTTCSRQFTSSSSMKGSCGIGGGIGGGSSRISSVLAGGSCRAPSTYGGGLSVSSSRFSSGGAYGLGGGYGGGFSSSSSSFGSGFGGGYGGGLGAGLGGGFGGGFAGGDGLLVGSEKVTMQNLNDRLASYLDKVRALEEANADLEVKIRDWYQRQRPAEIKDYSPYFKTIEDLRNKILTATVDNANVLLQIDNARLAADDFRTKYETELNLRMSVEADINGLRRVLDELTLARADLEMQIESLKEELAYLKKNHEEEMNALRGQVGGDVNVEMDAAPGVDLSRILNEMRDQYEKMAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEDAHLSSSQFSSGSQSSRDVTSSSRQIRTKVMDVHDGKVVSTHEQVLRTKN

(19) United States
(12) Patent Application Publication
(10) Pub. No.: US 2018/0230436 A1
(43) Pub. Date: Aug. 16, 2018

(54) METHODS OF MANUFACTURING KERATIN PRODUCTS AND USES THEREOF

(71) Applicants: Garrett VYGANTAS, San Francisco, CA (US); Bibhash MUKHOPADHYAY, Highland Park, NJ (US); Darryl CARTER, Owings Mills, MD (US)

(72) Inventors: Garrett VYGANTAS, San Francisco, CA (US); Bibhash MUKHOPADHYAY, Highland Park, NJ (US); Darryl CARTER, Owings Mills, MD (US)

(21) Appl. No.: 15/525,241

(22) PCT Filed: Oct. 8, 2015
(86) PCT No.: PCT/US2015/054760

§ 371 Date: May 8, 2017

Related U.S. Application Data
(60) Provisional application No. 62/062,388, filed on Oct. 10, 2014.

(51) Int. Cl. C12N 5/074; C12N 5/071; C12P 21/02

(52) U.S. Cl. CPC C12N 5/0696; C12N 5/0629; C12P 21/02; C12N 2506/45; C12N 2501/385; C12N 2501/602; C12N 2501/155

(57) ABSTRACT

Methods and compositions are provided for producing synthetically-derived keratin compositions and keratin products.

### CROSS REFERENCE TO RELATED APPLICATIONS

This application is the National Stage Entry of International Application No. PCT/US2015/054760, filed Oct. 8, 2015, which claims the benefit of U.S. Provisional Application No. 62/062,388, filed on Oct. 10, 2014, both of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Oct. 31, 2017, is named “44542701831_SL.txt” and is 31,632 bytes in size.

### SUMMARY OF THE INVENTION

Disclosed herein, in certain embodiments, are methods for producing a rhinoceros keratinocyte comprising contacting one or more embryoid bodies with one or more solutions to produce a rhinoceros keratinocyte, wherein: the one or more embryoid bodies are formed from a rhinoceros induced pluripotent stem cell (rhino iPSC); and the one or more solutions comprise one or more nutrients, one or more growth factors, or a combination thereof. In some embodiments, the one or more solutions comprise embryonic stem cell (ESC) medium. In some embodiments, the ESC medium does not comprise leukemia inhibitory factor (LIF). In some embodiments, the one or more solutions comprise a first solution and a second solution. In some embodiments, the one or more embryoid bodies are contacted with the first solution and the second solution sequentially. In some embodiments, the one or more embryoid bodies are contacted with the first solution and the second solution simultaneously. In some embodiments, the one or more nutrients are selected from the group comprising retinoic acid, retinol, amino acids, purines, pyrimidines, vitamins, glucose, and inorganic ions. In some embodiments, the one or more nutrients comprise retinoic acid. In some embodiments, the one or more growth factors are selected from the group comprising bone-morphogenetic protein-4 (BMP-4), BMP-2, BMP-6, BMP-7, epidermal growth factor (EGF), fibroblast growth factor 2 (FGF-2), granulocyte colony stimulating factor (G-CSF), granulocyte/macrophage colony stimulating factor (GM-CSF), insulin like growth factor I (IGF-I), and vascular endothelial growth factor (VEGF). In some embodiments, the one or more growth factors comprise bone-morphogenetic protein-4 (BMP-4). In some embodiments, the methods further comprise culturing the rhinoceros keratinocyte on a coated cell culture surface. In some embodiments, the methods further comprise passaging the rhinoceros keratinocytes on a coated cell culture surface one or more times. In some embodiments, the coated cell culture surface is coated with collagen, fibronectin, poly-D-lysine, gelatin, laminin, hydrogel, extracellular matrix (ECM) or a combination thereof. In some embodiments, the collagen is collagen I, collagen IV, or a combination thereof. In some embodiments, the coated cell culture surface is coated with collagen IV. In some embodiments, the methods further comprise culturing the one or more embryoid bodies or the rhinoceros keratinocytes in embryonic stem cell (ESC) medium. In some embodiments, the ESC medium does not comprise leukemia inhibitory factor (LIF). In some embodiments, the methods further comprise culturing the rhinoceros keratinocytes in keratinocyte serum-free medium (KSFM). In some embodiments, the methods further comprise differentiating the rhinoceros keratinocyte to produce a differentiated keratinocyte. In some embodiments, differentiating the rhinoceros keratinocyte comprises contacting the rhinoceros keratinocyte with a culture medium comprising calcium (Ca²⁺). In some embodiments, differentiating the rhinoceros keratinocyte comprises increasing the calcium (Ca²⁺) concentration in the culture medium. In some embodiments, increasing the calcium (Ca²⁺) concentration comprises at least doubling the calcium (Ca²⁺) concentration in the culture medium. In some embodiments, the concentration of calcium (Ca²⁺) in the culture medium is between about 0.005 mM to about 0.60 mM. In some embodiments, the concentration of calcium (Ca²⁺) in the culture medium is at least about 0.02 mM. In some embodiments, the concentration of calcium (Ca²⁺) in the culture medium is about 0.35 mM. In some embodiments, the rhino iPSC expresses POU5F1, SOX2, NANOG, or a combination thereof. In some embodiments, the rhinoceros keratinocyte expresses Krt14, Krt10, or a combination thereof. In some embodiments, the differentiated keratinocyte expresses Krt1, loricrin, or a combination thereof.

Disclosed herein, in certain embodiments, are methods of producing a composition comprising keratin, the method comprising transfecting an induced pluripotent stem cell (iPSC) with one or more genes encoding keratin. In some embodiments, the iPSC is further transfected with one or more genes encoding melanin. In some embodiments, the melanin comprises eumelanin, pheomelanin, or a combination thereof. In some embodiments, the composition further comprises melanin. In some embodiments, the percent composition of melanin is at least about 0.5% w/w of the total composition. In some embodiments, the percent composition of melanin is at least about 1% w/w of the total composition. In some embodiments, the percent composition of melanin is at least about 5% w/w of the total composition. In some embodiments, the composition further comprises calcium. In some embodiments, the percent composition of calcium is at least about 0.5% w/w of the total composition. In some embodiments, the percent composition of calcium is at least about 1% w/w of the total composition. In some embodiments, the percent composition of calcium is at least about 5% w/w of the total composition. In some embodiments, the composition further comprises water. In some embodiments, the percent composition of water is at least about 10% w/w of the total composition. In some embodiments, the composition of the water is at least about 20% w/w of the total composition. In some embodiments, the composition of the water is at least about 40% w/w of the total composition. In some embodiments, the iPSC is based on or derived from a rhinoceros cell. In some embodiments, the iPSC is based on or derived from an epidermal cell. In some embodiments, the iPSC is transfected with two or more genes encoding keratin. In some embodiments, the iPSC is transfected with three or more genes encoding keratin. In some embodiments, the keratin comprises an amino acid sequence that is at least about 50% identical to SEQ ID NOs: 5-8. In some embodiments, the keratin is an alpha keratin. In some embodiments, the one or more genes is KRT1, KRT5, KRT10, KRT14, or a combination thereof. In some embodiments, the amino acid sequence of keratin comprises one or more cysteine residues. In some embodiments, at least about 5% of the amino acid residues of the amino acid sequence of keratin are cysteine residues. In some embodiments, at least about 6% of the amino acid residues of the amino acid sequence of keratin are cysteine residues. In some embodiments, at least about 8% of the amino acid residues of the amino acid sequence of keratin are cysteine residues. In some embodiments, the amino acid sequence of keratin comprises one or more cysteine residues. In some embodiments, at least about 5% of the amino acid residues of the amino acid sequence of keratin are glycine residues. In some embodiments, at least about 6% of the amino acid residues of the amino acid sequence of keratin are glycine residues. In some embodiments, at least about 8% of the amino acid residues of the amino acid sequence of keratin are glycine residues. In some embodiments, the keratin comprises one or more disulfide bonds formed between two or more amino acid residues of keratin. In some embodiments, the keratin comprises one or more hydrogen bonds formed between two or more amino acid residues of keratin. In some embodiments, the percent composition of keratin is at least about 30% w/w of the total composition. In some embodiments, the percent composition of keratin is at least about 40% w/w of the total composition. In some embodiments, the percent composition of keratin is at least about 50% w/w of the total composition. In some embodiments, the methods further comprise culturing the iPSC. In some embodiments, the iPSC is cultured for at least about 24 hours. In some embodiments, culturing the iPSC comprises producing two or more keratin-producing iPSCs. In some embodiments, the methods further comprise contacting the iPSCs with one or more scaffolds. In some embodiments, the iPSCs are contacted with the scaffold prior to culturing the iPSC. In some embodiments, the iPSCs are contacted with the scaffold after culturing the iPSC. In some embodiments, contacting the iPSCs with one or more scaffolds comprises contacting the keratin-producing iPSCs with the one or more scaffolds. In some embodiments, the one or more scaffolds comprise a biorubber. In some embodiments, the one or more scaffolds are biodegradable. In some embodiments, the one or more scaffolds comprise a biodegradable plastic. In some embodiments, the one or more scaffolds comprise a biodegradable polymer. In some embodiments, the one or more scaffolds are flexible. In some embodiments, the one or more scaffolds are elastic. In some embodiments, the one or more scaffolds comprise glycerol. In some embodiments, the one or more scaffolds are conical. In some embodiments, the one or more conical scaffolds are in the shape of a horn. In some embodiments, the methods further comprise purifying the composition. In some embodiments, the methods further comprise shaping the composition. In some embodiments, the methods further comprise shaping the composition into a horn-like structure. In some embodiments, the methods further comprise shaping the composition into a conical-like structure. In some embodiments, the methods further comprise contacting the iPSC with one or more biominerals. In some embodiments, the methods further comprise contacting the iPSC with one or more biominerals. In some embodiments, the one or more biominerals comprise silicates, carbonates, calcium phosphates, gold, copper, iron, phosphates, or any combination thereof.

Disclosed herein, in certain embodiments, are methods of producing a composition comprising keratin, melanin and calcium, the method comprising transfecting an induced pluripotent stem cell (iPSC) with one or more genes encoding keratin.

Disclosed herein, in certain embodiments, are methods of producing a composition comprising keratin, the method comprising contacting one or more embryoid bodies with one or more solutions to produce a rhinoceros keratinocyte, wherein: the one or more embryoid bodies are formed from a rhinoceros induced pluripotent stem cell (rhino iPSC); and the one or more solutions comprise one or more nutrients, one or more growth factors, or a combination thereof.

Disclosed herein, in certain embodiments, are compositions comprising keratin, wherein the composition is produced by a method comprising transfecting an induced pluripotent stem cell (iPSC) with one or more genes encoding keratin.

Disclosed herein, in certain embodiments, are compositions comprising keratin, melanin and calcium, wherein the composition is produced by a method comprising transfecting an induced pluripotent stem cell (iPSC) with one or more genes encoding keratin.

Disclosed herein, in certain embodiments, are compositions comprising keratin, wherein the composition is produced by a method comprising contacting one or more embryoid bodies with one or more solutions to produce a rhinoceros keratinocyte, wherein: the one or more embryoid bodies are formed from a rhinoceros induced pluripotent stem cell (rhino iPSC); and the one or more solutions comprise one or more nutrients, one or more growth factors, or a combination thereof. In some embodiments, the keratin is keratin 1, keratin 5, keratin 10, keratin 14, or a combination thereof. In some embodiments, the keratin is alpha-keratin. In some embodiments, the one or more genes are KRT1, KRT5, KRT10, KRT14 or a combination thereof. In some embodiments, the amino acid sequence of keratin comprises one or more cysteine residues. In some embodiments, at least about 5% of the amino acid residues of the amino acid sequence of keratin are cysteine residues. In some embodiments, at least about 6% of the amino acid residues of the amino acid sequence of keratin are cysteine residues. In some embodiments, at least about 8% of the amino acid residues of the amino acid sequence of keratin are cysteine residues. In some embodiments, the amino acid sequence of keratin comprises one or more cysteine residues. In some embodiments, at least about 5% of the amino acid residues of the amino acid sequence of keratin are glycine residues. In some embodiments, at least about 6% of the amino acid residues of the amino acid sequence of keratin are glycine residues. In some embodiments, at least about 8% of the amino acid residues of the amino acid sequence of keratin are glycine residues. In some embodiments, the keratin comprises one or more disulfide bonds formed between two or more amino acid residues of keratin. In some embodiments, the keratin comprises one or more hydrogen bonds formed between two or more amino acid residues of keratin. In some embodiments, the percent composition of keratin is at least about 30% w/w of the total composition. In some embodiments, the percent composition of keratin is at least about 40% w/w of the total composition. In some embodiments, the percent composition of keratin is at least about 50% w/w of the total composition. In some embodiments, the iPSC is further transfected with one or more genes encoding melanin. In some embodiments, the melanin comprises eumelanin, pheomelanin, or a combination thereof. In some embodiments, the compositions further comprise melanin. In some embodiments, the percent composition of melanin is at least about 0.5% w/w of the total composition. In some embodiments, the percent composition of melanin is at least about 1% w/w of the total composition. In some embodiments, the percent composition of melanin is at least about 5% w/w of the total composition. In some embodiments, the composition further comprises calcium. In some embodiments, the percent composition of calcium is at least about 0.5% w/w of the total composition. In some embodiments, the percent composition of calcium is at least about 1% w/w of the total composition. In some embodiments, the percent composition of calcium is at least about 5% w/w of the total composition. In some embodiments, the composition further comprises water. In some embodiments, the percent composition of water is at least about 10% w/w of the total composition. In some embodiments, the composition of the water is at least about 20% w/w of the total composition. In some embodiments, the composition of the water is at least about 40% w/w of the total composition. In some embodiments, the keratin forms a two-stranded molecule. In some embodiments, the keratin forms one or more intermediate filaments. In some embodiments, the density of the intermediate filaments is at least about 5 mm⁻². In some embodiments, the density of the intermediate filament is at least about 6 mm⁻². In some embodiments, the density of the intermediate filament is at least about 7 mm⁻². In some embodiments, the diameter of the intermediate filament is at least about 50 μm. In some embodiments, the diameter of the intermediate filament is at least about 70 μm. In some embodiments, the diameter of the intermediate filament is at least about 100 μm. In some embodiments, the intermediate filaments are embedded in a matrix. In some embodiments, the matrix is an amorphous protein matrix. In some embodiments, the matrix comprises a keratin matrix. In some embodiments, the matrix comprises a non-crystalline keratin matrix. In some embodiments, the keratin surrounds a core. In some embodiments, the core is a hair-like core. In some embodiments, the core is a non-fibrous core. In some embodiments, the core is solid. In some embodiments, the composition is in the shape of a horn. In some embodiments, the compositions further comprise cholesterol, taurine, hexosamine, phospholipid, or a combination thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 depicts a schematic of synthesizing a keratinocyte.

FIG. 2 depicts rhinoceros horns.

FIG. 3A depicts elephant tusks.

FIG. 3B-3C depict decorative uses of elephant tusks.

FIG. 4A depicts a dagger handle comprising rhino horn.

FIG. 4B depicts gun handles comprising ivory.

### DETAILED DESCRIPTION OF THE INVENTION

The use of animal products, such as rhinoceros horn and elephant tusks, for decorative and/or medicinal purposes have resulted in near extinction of these animals. For example, rhinoceros horn is often used for the handles of curved daggers called “jambiya.” Rhino horns have also been carved into ceremonial cups, buttons, belt buckles, hair pins, drawer handles, and paperweights. In addition to their decorative uses, rhinoceros horns are often used in traditional medicine systems of many Asian countries. The ability to synthetically produce keratin products with similar compositions and appearance may provide an alternative to the naturally produced animal products. The present invention provides methods and compositions for the manufacture of keratin products.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims. Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural references unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Disclosed herein, are methods of manufacturing compositions comprising keratin. Further disclosed herein, are methods of manufacturing compositions comprising (a) keratin; and (b) melanin, calcium, water, or any combination thereof. Generally, the method comprises (a) contacting one or more cells with one or more solutions to produce a differentiated cell; and (b) inducing the differentiated cell to produce keratin, thereby producing a composition comprising keratin. In some embodiments, the method further comprises terminally differentiating the differentiated cell to produce a terminally differentiated cell.

In some embodiments, methods of manufacturing keratin compositions comprise contacting one or more embryoid bodies with one or more solutions to produce a keratinocyte, wherein (a) the one or more embryoid bodies are formed from an induced pluripotent stem cell (iPSC); and (b) the one or more solutions comprise one or more nutrients, growth factors, or a combination hereof.

Alternatively, the method of manufacturing a keratin composition comprises transfecting pluripotent cell with one or more genes encoding keratin. In some embodiments, the pluripotent cell is an induced pluripotent stem cell (iPSC). In some embodiments, the pluripotent cell is a pluripotent stem cell. In some embodiments, the pluripotent cell is transfected with two or more genes encoding keratin.

In some embodiments, the one or more cells are stem cells. In other embodiments, the one or more stem cells are embryonic stem cells (ESCs). Alternatively, or additionally, the one or more cells are pluripotent cells. The one or more cells may be pluripotent stem cells. In some embodiments, the one or more cells comprise embryoid bodies.

In some embodiments, embryoid bodies (EBs) are three-dimensional aggregates of pluripotent stem cells. The pluripotent cell types that comprise embryoid bodies may include, but are not limited to, embryonic stem cells (ESCs) derived from the blastocyst stage of embryos from mouse (mESC), primate, and human (hESC) sources. Additionally, EBs may be formed from embryonic stem cells derived through alternative techniques, including somatic cell nuclear transfer or the reprogramming of somatic cells to yield induced pluripotent stem cells (iPS or iPSC). Similar to ESCs cultured in monolayer formats, ESCs within embryoid bodies may undergo differentiation and cell specification along the three germ lineages, endoderm, ectoderm, and mesoderm, which comprise most somatic cell types. In some embodiments, the EBs are from an epidermal stem cell. Alternatively, or additionally, the EBs are formed from an iPSC. In some embodiments, the EBs are formed from a rhinoceros epidermal stem cell. Alternatively, or additionally, the EBs are formed from a rhinoceros iPSC.

In some embodiments, the pluripotent stem cell expresses POU5F1, SOX2, NANOG, or a combination thereof. In some embodiments, the pluripotent cell expresses POU5F1, SOX2, NANOG, or a combination thereof. In some embodiments, the iPSC expresses POU5F1, SOX2, NANOG, or a combination thereof.

In some embodiments, the one or more cells are from a rhinoceros. In other embodiments, the one or more cells are from an elephant. Alternatively, the one or more cells are from a mammal The one or more cells may be from a sheep. The one or more cells may be from a warthog. The one or more cells may be from an animal with a horn. Alternatively, the one or more cells are from an animal with a tusk. In some embodiments, the one or more cells are from a human, ape, monkey,

In some embodiments, the differentiated cell is an epidermal cell. In some embodiments, the differentiated cell is a keratinocyte. The keratinocyte may be a corneocyte. Alternatively, or additionally, the differentiated cell is a basal cell or basal keratinocyte. The differentiated cell may be a rhinoceros cell. In some embodiments, the method comprises contacting the one or more cells with one or more solutions to produce a keratinocyte. The keratinocyte may be a rhinoceros keratinocyte. In some embodiments, the keratinocyte expresses Krt14, Krt10, or a combination thereof. In some embodiments, the keratinocyte expresses keratin 5. In some embodiments, the keratinocyte expresses involucrin, loricrin, transglutaminase, filaggrin, caspase 14, or a combination thereof.

In some embodiments, the one or more solutions promote differentiation of the embryoid bodies. The one or more solutions may be an embryoid body differentiation solution. The one or more solutions may be a differentiation solution. In some embodiments, the one or more solutions comprise stem cell factor (SCF or kit ligand (KL)). SCF may exist in two different forms generated by alternative splicing: a longer one, designated KL-1, is a transmembrane protein of 248 amino acids that can be cleaved by proteolysis to release soluble SCF; and a shorter one, designated KL-2, is a transmembrane protein of 220 amino acids. In some embodiments, the mature KL-1 protein is about 45 kDa in size. The KL-1 protein may be processed by proteolytic cleavage and modified by glycosylation to generate smaller fragments of 40, 35, and 24 kDa. The KL-2 protein products may consist of 32 and 28 kDa forms.

Alternatively, or additionally, the one or more solutions comprise vasoactive intestinal peptide (VIP). In some embodiments, the one or more solutions comprise calcium. In some embodiments, the one or more solutions comprise one or more vitamins In some embodiments, the one or more vitamins are vitamin D3. In some embodiments, the one or more solutions comprise one or more cathepsins. In some embodiments, the one or more cathepsins are cathepsin E. In some embodiments, the one or more solutions comprises hydrocortisone.

In some embodiments, the methods disclosed herein further comprise activating one or more transcription factors in the keratinocyte or pluripotent cell. In some embodiments, transcription factor is a TALE homeodomain transcription factor.

In some embodiments, the method of manufacturing compositions comprising keratin comprises (a) contacting one or more embryoid bodies with one or more solutions to produce a keratinocyte; and (b) inducing the keratinocyte to secrete keratin, thereby producing a composition comprising keratin. In some embodiments, inducing the keratinocyte to secrete keratin comprises differentiating the keratinocyte. Differentiating the keratinocyte may comprise withdraw of the keratinocyte from the cell cycle, expression of epidermal differentiation markers, and/or migration of the keratinocyte toward the suprabasal layers. Migration of the keratinocyte toward the suprabasal layer may comprise incorporation of the keratinocyte to the stratum spinosum, stratum granulosum and/or stratum corneum. Differentiating the keratinocyte may comprise producing a corneocyte from the keratinocyte.

Differentiating the keratinocyte may comprise producing a differentiated keratinocyte. The differentiated keratinocyte may be a corneocyte. The differentiated keratinocyte may be devoid of one or more intracellular organelles. The differentiated keratinocyte may be devoid of all intracellular organelles. The differentiated keratinocyte may be devoid of a nucleus.

Differentiation of the keratinocyte may comprise contacting the keratinocyte with one or more solutions. In some embodiments, inducing the differentiated cell to produce keratin comprises contacting the differentiated cell with one or more solutions to produce a terminally differentiated cell. The one or more solutions may be a keratinocyte differentiation solution. The one or more solutions may be a terminal differentiation solution. In some embodiments, the one or more solutions comprise one or more vitamins, metals, proteases, steroids, transcription factors, or a combination thereof. In some embodiments, the one or more solutions comprise calcium, vitamin D3, cathepsin E, TALE homeodomain transcription factors, hydrocortisone, or any combination thereof.

In some embodiments, vitamins include, but are not limited to, vitamin A (retinol), vitamin B1 (thiamine), vitamin C (ascorbic acid), vitamin D (calciferol), vitamin D3 (cholecalciferol), vitamin B2 (riboflavin), vitamin E (tocopherol), vitamin B12 (cobalamins), vitamin K1, vitamin B5 (pantothenic acid), vitamin B7 (biotin), vitamin B6 (pyridoxine), vitamin B3 (niacin), and vitamin B9 (folic acid). In some embodiments, the one or more solutions comprise vitamin D In some embodiments, the one or more solutions comprise vitamin D3.

In some embodiments, metals include, but are not limited to, alkali metals, alkaline earth metals, transition metals, post-transition metals, lanthanides, and actinides. In some embodiments, the one or more solutions comprise an alkali metal. Examples of alkali metals include, but are not limited to, lithium, sodium, potassium, rubidium, caesium and francium.

In some embodiments, the one or more solutions comprise an alkaline earth metal. Examples of alkaline earth metals include, but are not limited to, beryllium, magnesium, calcium, strontium, barium, radium. In some embodiments, the one or more solutions comprise calcium.

In some embodiments, the one or more solutions comprise a transition metal. Transition metals include, but are not limited to, zinc, molybdenum, cadmium, scandium, titanium, vanadium, chromium, manganese, iron, cobalt, nickel, copper, yttrium, zirconium, niobium, technetium, ruthenium, rhodium, palladium, silver, hafnium, tantalum, tungsten, rhenium, osmium, iridium, platinum, gold, mercury, rutherfordium, dubnium, seaborgium, bohrium, hassium, and copernicium.

In some embodiments, the one or more solutions comprise a post-transition metal. Post-transition metals include, but are not limited to, aluminium, gallium, indium, tin, thallium, lead, bismuth, and polonium.

In some embodiments, the one or more solutions comprise a lanthanide. Lanthanides include, but are not limited to, lanthanum, cerium, praseodymium, neodymium, promethium, samarium, europium, gadolinium, terbium, dysprosium, holmium, erbium, thulium, ytterbium, and lutetium.

In some embodiments, the one or more solutions comprise an actinide. Actinides include, but are not limited to, actinium, thorium, protactinium, uranium, neptunium, plutonium, americium, curium, berkelium, californium, einsteinium, fermium, mendelevium, nobelium, and lawrencium.

In some embodiments, the one or more solutions comprise a protease. Examples of proteases include, but are not limited to, serine proteases, threonine proteases, cysteine proteases, aspartate proteases (aspartic proteases), glutamic proteases, and metalloproteases. In some embodiments, the one or more solutions comprise an aspartatic protease. In some embodiments, the aspartic proteases comprise pepsins, cathepsins, and renins.

In some embodiments, the one or more solutions comprise a cathepsin. Cathepsins include, but are not limited to, cathepsin A, cathepsin B, cathepsin C, cathepsin D, cathepsin E, cathepsin F, cathepsin G, cathepsin H, cathepsin K, cathepsin L1, cathepsin L2 (cathepsin V, cathepsin O, cathepsin S, cathepsin W, and Cathepsin Z (or cathepsin X). In some embodiments, the one or more solutions comprise cathepsin E.

In some embodiments, the one or more solutions comprise a steroid. The steroid may be a steroid hormone. Steroid hormones may comprise steroids which bind to glucocorticoids, mineralocorticoids, androgens, estrogens, and progestogens. Steroid hormones include, but are not limited to, alclometasone, prednisone, dexamethasone, triamcinolone, fludrocortisone, apoptone, oxandrolone, oxabolone, testosterone, nandrolone (also known as anabolic steroids), diethylstilbestrol (DES), danazol, norethindrone, medroxyprogesterone acetate, and 17-Hydroxyprogesterone caproate. In some embodiments, the one or more solutions comprise hydrocortisone.

In some embodiments, the one or more solutions comprise a transcription factor. In some embodiments, the transcription factor is a basic helix-loop-helix transcription factor, basic-leucine zipper (bZIP) transcription factor, GCC box transcription factor, helix-turn-helix transcription factor, homeodomain transcription factor, lambda repressor-like transcription factor, srf-like (serum response factor) transcription factor, winged helix transcription factor, and/or zinc finger transcription factor. In some embodiments, the one or more solutions comprise a TALE homeodomain transcription factor.

In some embodiments, differentiation comprises culturing the cells (e.g., keratinocytes, differentiated cells) in in a calcium gradient. The calcium gradient may comprise increasing concentrations of calcium. In some embodiments, differentiation comprises increasing the calcium concentration of the culture medium. In some embodiments, increasing the calcium concentration comprises at least doubling the calcium concentration in the culture medium. In some embodiments, the calcium concentration in the culture medium is increased by at least about 5%, 10%, 15%, 20%, 25%, 30%, 35%, 40%, 45%, 50%, 55%, 60%, 65%, 70%, 75%, 80%, 85%, 90%, or 95% or more. In some embodiments, the calcium concentration in the culture medium is increased by at least about 1.5-fold, 2-fold, 2.5-fold, 3-fold, 3.5-fold, 4-fold, 4.5-fold, or 5-fold. In some embodiments, the calcium concentration in the culture medium is at least about 0.001 mM. In some embodiments, the calcium concentration in the culture medium is at least about 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, or 1 mM. In some embodiments, the calcium concentration in the culture medium is between about 0.005 mM to about 0.60 mM. In some embodiments, the concentration of calcium in the culture medium is at least about 0.02 mM. In some embodiments, the concentration of calcium in the culture medium is at least about 0.35 mM.

Differentiation may comprise altering the intracellular calcium concentration of the cells (e.g., keratinocytes, differentiated cells). In some embodiments, intracellular calcium concentration can be used to distinguish the cell from the terminally differentiated cell. In some embodiments, the intracellular calcium concentration of the terminally differentiated cell is higher than the intracellular calcium concentration of the cell (e.g., keratinocyte, differentiated cell). In some embodiments, the intracellular calcium concentration of the terminally differentiated cell is at least about 5%, 10%, 15%, 20%, 25%, 30%, 35%, 40%, 45%, 50%, 55%, 60%, 65%, 70%, 75%, 80%, 85%, 90%, or 95% higher than the intracellular calcium concentration of the cell (e.g., keratinocyte, differentiated cell). In some embodiments, the intracellular calcium concentration of the terminally differentiated cell is at least about 1.5-fold, 2-fold, 2.5-fold, 3-fold, 3.5-fold, 4-fold, 4.5-fold, or 5-fold greater than the intracellular calcium concentration of the cell (e.g., keratinocyte, differentiated cell).

In some embodiments, the cell (e.g., keratinocyte, differentiated cell) can be distinguished from the terminally differentiated cell by one or more markers. The one or more markers may comprise keratin, involucrin, loricrin, transglutamase, filaggrin, and caspase. In some embodiments, keratin comprises keratin 1, keratin 5, keratin 10, keratin 14, or any combination thereof. In some embodiments, caspase comprises caspase 14. Alternatively, or additionally, the one or more markers comprise Krt1, loricrin, or a combination thereof. In some embodiments, expression of the one or more markers in decreased in the terminally differentiated cell as compared to the cell (e.g., keratinocyte, differentiated cell). In some embodiments, expression of Krt14 and/or Krt10 is decreased in the terminally differentiated cell as compared to the cell (e.g., keratinocyte, differentiated cell). In some embodiments, expression of the one or more markers in increased in the terminally differentiated cell as compared to the cell (e.g., keratinocyte, differentiated cell). In some embodiments, expression of Krt1 and/or loricrin is increased in the terminally differentiated cell as compared to the cell (e.g., keratinocyte, differentiated cell).

In some embodiments, the method further comprises culturing the cells (e.g., stem cells, iPSC, differentiated cells, and keratinocytes). The cells may be cultured for at least about 6, 12, 18, 24, 30, 36, 42, 48, 54, 60, 66, 72 or more hours. The cells may be cultured for at least about 1, 2, 3, 4, 5, 6, 7, 8, 9, 10, 11, 12, 13, 14 or more days. Culturing the cells may comprise culturing the cells in one or more solutions. The one or more solutions may comprise transcription factors, vitamins, growth factors, and toxins. The one or more solutions may comprise transcription factor p63. The one or more solutions may comprise vitamin A or its analogues. The one or more solutions may comprise epidermal growth factor and/or tumor growth factor alpha. The one or more solutions may comprise cholera toxin.

In some embodiments, the method further comprises culturing the cells (e.g., stem cells, iPSC, differentiated cells, keratinocytes) with one or more other cell types. The one or more other cell types may comprise melanocytes and/or Langerhans cells.

In some embodiments, the method further comprises culturing the cells (e.g., stem cells, iPSC, differentiated cells, keratinocytes) on a coated cell culture surface. In some embodiments, the coated cell culture surface is coated with collagen, fibronectin, poly-D-lysine, gelatin, laminin, hydrogel, extracellular matrix (ECM), or a combination thereof. In some embodiments, collagen is collagen I, collagen IV, or a combination thereof.

In some embodiments, the method further comprises passaging the cells (e.g., stem cells, iPSC, differentiated cells, keratinocytes) on the coated cell culture surface 1 or more times. The method may further comprise passaging the cells on the coated cell culture surface 2, 3, 4, 5, 6, 7, 8, 9, 10 or more times.

In some embodiments, the method further comprises culturing the cells (e.g., stem cells, iPSC, differentiated cells, keratinocytes) in embryonic stem cell (ESC) medium. In some embodiments, the ESC medium does not comprise leukemia inhibitory factor (LIF). In some embodiments, the method further comprises culturing the cells (e.g., stem cells, iPSC, differentiated cells, keratinocytes) in keratinocyte serum-free medium (KSFM).

In some embodiments, the method further comprises transfecting the cells (e.g., stem cells, iPSC, differentiated cells, keratinocytes) with one or more nucleic acids encoding melanin. In some embodiments, the method further comprises transfecting the cells (e.g., stem cells, iPSC, differentiated cells, keratinocytes) with two or more nucleic acids encoding melanin. In some embodiments, the method further comprises transfecting the cells (e.g., stem cells, iPSC, differentiated cells, keratinocytes) with 3, 4, 5, 6, 7, 8, 9, 10 or more nucleic acids encoding melanin. In some embodiments, melanin comprises eumelanin, pheomelanin, or a combination thereof.

In some embodiments, the method further comprises contacting the cells (e.g., stem cells, iPSC, differentiated cells, keratinocytes) with one or more biominerals. In some embodiments, the one or more biominerals comprise silicates, carbonates, calcium phosphates, gold, copper, iron, phosphates, or any combination thereof.

### Scaffolds

In some embodiments, the method further comprises contacting the cells (e.g., stem cells, iPSC, differentiated cells, keratinocytes) with one or more scaffolds. In some embodiments, the cells are contacted with the scaffold prior to culturing the cells. In other embodiments, the cells are contacted with the scaffold after culturing the cells. In other embodiments, the cells are contacted with the scaffold prior to differentiation. Alternatively, the cells are contacted with the scaffold after differentiation. In other embodiments, the cells are contacted with the scaffold prior to terminal differentiation. Alternatively, the cells are contacted with the scaffold after terminal differentiation. In some embodiments, the cells are contacted with the scaffold prior to inducing the cells to produce keratin. In other embodiments, the cells are contacted with the scaffold after inducing the cells to produce keratin.

In some embodiments, the one or more scaffolds are biodegradable. In some embodiments, the one or more scaffolds are flexible. In other embodiments, the one or more scaffolds are elastic. Alternatively, the one or more scaffolds are stiff, rigid, inelastic, and/or inflexible.

In some embodiments, the one or more scaffolds comprise a biodegradable rubber. In some embodiments, the biodegradable rubber is hard and/or brittle. In other embodiments, the biodegradable rubber is inflexible. The biodegradable rubber may be rigid, stiff and/or inelastic.

In some embodiments, the one or more scaffolds comprise a biorubber. In some embodiments, the biorubber is white and/or opaque. The biorubber may be elastic and/or flexible.

In some embodiments, the one or more scaffolds comprise a biodegradable plastic. In some embodiments, the biodegradable plastic comprises an aliphatic polyester. Examples of biodegradable plastics include, but are not limited to, polyhydroxyalkanoates (PHAs) like the poly-3-hydroxybutyrate (PHB), polyhydroxyvalerate (PHV) and polyhydroxyhexanoate (PHH); polylactic acid (PLA); polybutylene succinate (PBS), polycaprolactone (PCL); polyanhydrides; polyvinyl alcohol and cellulose esters like cellulose acetate and nitrocellulose and their derivatives (celluloid).

In other embodiments, the one or more scaffolds comprise a biodegradable polymer. In some embodiments, biodegradable polymers are polymers that break down and lose their initial integrity. In some embodiments, the biodegradable polymer comprises 3-hydroxypropionic acid. In other embodiments, the biodegradable polymer comprises polylactic acid.

In some embodiments, the one or more scaffolds comprise glycerol.

In some embodiments, the one or more scaffolds comprise a 3D matrix. In some embodiments, the scaffolds comprise polymeric scaffolds. In some embodiments, the scaffolds comprise bioscaffolds or biomimetic scaffolds. In other embodiments, the scaffolds comprise extra-cellular matrix scaffolds. In some embodiments, the scaffolds comprise composite scaffolds. In other embodiments, the scaffolds comprise nanofiber scaffolds. In some embodiments, the scaffolds comprise collagen scaffolds.

In some embodiments, the one or more scaffolds are conical. In some embodiments, the one or more conical scaffolds are in the shape of a horn. In other embodiments, the one or more conical scaffolds are in the shape of a tusk.

In some embodiments, the one or more scaffolds are produced by 3D printing.

### Cells for Manufacturing Keratin

Further disclosed herein are one or more cells for manufacturing a keratin composition. In some embodiments, the one or more cells comprise a pluripotent cell. In some embodiments, the pluripotent cell is a stem cell. In other embodiments, the pluripotent cell is an induced pluripotent stem cell (iPSC).

Further disclosed herein are embryoid bodies for manufacturing a keratin composition. In some embodiments, the embryoid cells are formed from a pluripotent cell. In certain embodiments, the embryoid cells are formed from a rhinoceros pluripotent cell. In certain embodiments, the embryoid cells are formed from a rhinoceros induced pluripotent stem cell (rhino iPSC). In other embodiments, the embryoid cells are formed from an elephant pluripotent cell. In certain embodiments, the embryoid cells are formed from an elephant induced pluripotent stem cell (elephant iPSC).

Further disclosed herein are keratinocytes for manufacturing a keratin composition. In some embodiments, the keratinocytes are produced from differentiated pluripotent cells. In certain embodiments, the keratinocytes are produced from differentiated induced pluripotent stem cells. In other embodiments, the keratinocytes are produced from differentiated embryoid cells.

Further disclosed herein are terminally differentiated cells for manufacturing a keratin composition. In some embodiments, the terminally differentiated cells are formed from terminally differentiated keratinocytes. In some embodiments, the terminally differentiated cells are corneocytes.

In some embodiments, the one or more cells for manufacturing a keratin composition are transfected with one or more nucleic acids encoding one or more keratins. In some embodiments, the one or more cells for manufacturing a keratin composition are transfected with one or more nucleic acids encoding two or more keratins. In some embodiments, the one or more cells for manufacturing a keratin composition are transfected with three or more nucleic acids encoding two or more keratins. In some embodiments, the one or more cells for manufacturing a keratin composition are transfected with one or more nucleic acids encoding four or more keratins. In some embodiments, the one or more cells for manufacturing a keratin composition comprise one or more nucleic acids encoding one or more keratins. In some embodiments, the one or more cells for manufacturing a keratin composition comprise one or more keratins.

In some embodiments, the one or more keratins are encoded by a nucleic acid that is at least about 50% identical to SEQ ID NOs: 1-4. In some embodiments, the one or more keratins are encoded by a nucleic acid that is at least about 55%, 57%, 60%, 65%, 67%, 70%, 77%, 80%, 82%, 85%, 87%, 90%, 92%, 95%, or 97% identical to SEQ ID NOs: 1-4. In some embodiments, the one or more keratins are encoded by a nucleic acid that is at least about 55% identical to SEQ ID NOs: 1-4. In some embodiments, the one or more keratins are encoded by a nucleic acid that is at least about 60% identical to SEQ ID NOs: 1-4. In some embodiments, the one or more keratins are encoded by a nucleic acid that is at least about 70% identical to SEQ ID NOs: 1-4. In some embodiments, the one or more keratins are encoded by a nucleic acid that is at least about 75% identical to SEQ ID NOs: 1-4. In some embodiments, the one or more keratins are encoded by a nucleic acid that is at least about 85% identical to SEQ ID NOs: 1-4. In some embodiments, the one or more keratins are encoded by a nucleic acid that is at least about 90% identical to SEQ ID NOs: 1-4.

In some embodiments, the one or more keratins comprise Krt1, Krt14, Krt10, or any combination thereof. In some embodiments, the one or more keratins comprise an amino acid sequence that is at least about 50% identical to SEQ ID NOs: 5-8. In some embodiments, the one or more keratins comprise an amino acid sequence that is at least about 55%, 60%, 65%, 70%, 75%, 80%, 85%, 90%, 95% or more identical to SEQ ID NOs: 5-8. In some embodiments, the one or more keratins comprise an amino acid sequence that is at least about 70% identical to SEQ ID NOs: 5-8. In some embodiments, the one or more keratins comprise an amino acid sequence that is at least about 85% identical to SEQ ID NOs: 5-8. In some embodiments, the one or more keratins comprise an amino acid sequence that is at least about 95% identical to SEQ ID NOs: 5-8.

In some embodiments, the amino acid sequence of keratin comprises one or more cysteine residues. In some embodiments, at least about 5% of the keratin amino acid sequence comprises cysteine. In some embodiments, at least about 6% of the keratin amino acid sequence comprises cysteine. In some embodiments, at least about 8% of the keratin amino acid sequence comprises cysteine. In some embodiments, at least about 10% of the keratin amino acid sequence comprises cysteine. In some embodiments, at least about 12%, 15%, 17%, 20% or 25% of the keratin amino acid sequence comprises cysteine.

In some embodiments, at least about 5% of the keratin amino acid sequence comprises glycine. In some embodiments, at least about 6% of the keratin amino acid sequence comprises glycine. In some embodiments, at least about 8% of the keratin amino acid sequence comprises glycine. In some embodiments, at least about 10%, 12%, 15%, 17%, 20%, or 25% of the keratin amino acid sequence comprises glycine.

In some embodiments, the keratin comprises one or more disulfide bonds formed between two or more amino acid residues of keratin. In some embodiments, the keratin comprises 2, 3, 4, 5, 6, 7, 8, 9, 10 or more disulfide bonds formed between 2, 3, 4, 5, 6, 7, 8, 9, 10, 11, 12, 13, 14, 15, 16, 17, 18, 19, 20 or more amino acid residues of keratin. In some embodiments, the keratin comprises 3 or more disulfide bonds formed between 2 or more amino acid residues of keratin. In some embodiments, the keratin comprises 4 or more disulfide bonds formed between 3 or more amino acid residues of keratin. In some embodiments, the keratin comprises 6 or more disulfide bonds formed between 3 or more amino acid residues of keratin.

In some embodiments, the keratin comprises one or more hydrogen bonds formed between two or more amino acid residues of keratin. In some embodiments, the keratin comprises 2, 3, 4, 5, 6, 7, 8, 9, 10 or more hydrogen bonds formed between 2, 3, 4, 5, 6, 7, 8, 9, 10, 11, 12, 13, 14, 15, 16, 17, 18, 19, 20 or more amino acid residues of keratin. In some embodiments, the keratin comprises 2, 3, 4, 5, 6, 7, 8, 9, 10 or more hydrogen bonds formed between 2, 3, 4, 5, 6, 7, 8, 9, 10, 11, 12, 13, 14, 15, 16, 17, 18, 19, 20 or more amino acid residues of keratin. In some embodiments, the keratin comprises 3 or more hydrogen bonds formed between 2 or more amino acid residues of keratin. In some embodiments, the keratin comprises 4 or more hydrogen bonds formed between 3 or more amino acid residues of keratin. In some embodiments, the keratin comprises 6 or more hydrogen bonds formed between 3 or more amino acid residues of keratin.

In some embodiments, the percent composition of keratin is at least about 30% w/w of the total composition. In some embodiments, the percent composition of keratin is at least about 35% w/w of the total composition. In some embodiments, the percent composition of keratin is at least about 40% w/w of the total composition. In some embodiments, the percent composition of keratin is at least about 45% w/w of the total composition. In some embodiments, the percent composition of keratin is at least about 50% w/w of the total composition. In some embodiments, the percent composition of keratin is at least about 60%, 65%, 70%, 75%, 77%, 80%, 85%, 87%, 90%, 95%, or 97% w/w of the total composition.

In some embodiments, the one or more cells for manufacturing a keratin composition are further transfected with one or more nucleic acids encoding melanin. In some embodiments, the one or more cells for manufacturing a keratin composition comprise one or more nucleic acids encoding melanin. In some embodiments, melanin comprises eumelanin, pheomelanin, or a combination thereof.

### Keratin Compositions

Disclosed herein, in some embodiments, are compositions comprising keratin, wherein the compositions are produced by a method comprising transfecting an induced pluripotent stem cell (iPSC) with one or more genes encoding keratin.

Further disclosed herein, are compositions comprising keratin, wherein the compositions are produced by a method comprising contacting one or more embryoid bodies with one or more solutions to produce a rhinoceros keratinocyte, wherein (a) the one or more embryoid bodies are formed from a rhinoceros induced pluripotent stem cell (rhino iPSC); and (b) the one or more solutions comprise one or more nutrients, one or more growth factors, or a combination thereof.

Further disclosed herein, are compositions comprising keratin, melanin and calcium, wherein the composition is produced by a method comprising transfecting an induced pluripotent stem cell (iPSC) with one or more genes encoding keratin.

In some embodiments, keratin is alpha-keratin, keratin 5, keratin 10, keratin 13, keratin 14, keratin 16, keratin 17, keratin 34, keratin 36, keratin 40, keratin 73, keratin 77, keratin 82, or keratin 84 In some embodiments, the keratin is alpha-keratin, keratin 5, keratin 10, keratin 14. In some embodiments, the keratin is alpha-keratin. In some embodiments, the keratin is 10. In some embodiments, the keratin is 14. In some embodiments, the one or more genes are KRT1, KRT5, KRT10, KRT13, KRT14, KRT16, KRT17, KRT34, KRT36, KRT40, KRT73, KRT77, KRT82, or KRT84. In some embodiments, the one or more genes are KRT1, KRT5, KRT10, or KRT14.

In some embodiments, the amino acid sequence of keratin comprises one or more cysteine residues. In some embodiments, the amino acid sequence of keratin comprises 2, 3, 4, 5, 6, 7, 8, 9, 10, 11, 12, 13, 14, 15, 16, 17, 18, 19, 20 or more cysteine residues. In some embodiments, the amino acid sequence of keratin comprises 3 or more cysteine residues. In some embodiments, the amino acid sequence of keratin comprises 4 or more cysteine residues. In some embodiments, the amino acid sequence of keratin comprises 7 or more cysteine residues. In some embodiments, the amino acid sequence of keratin comprises 10 or more cysteine residues.

In some embodiments at least about 5% of the keratin amino acid sequence comprises cysteine. In some embodiments, at least about 6% of the keratin amino acid sequence comprises cysteine. In some embodiments, at least about 8% of the keratin amino acid sequence comprises cysteine. In some embodiments, at least about 10%, 12%, 15%, 17%, 20%, 22%, 25%, 27%, or 30% of the keratin amino acid sequence comprises cysteine.

In some embodiments, the amino acid sequence of keratin comprises one or more glycine residues. In some embodiments, the amino acid sequence of keratin comprises 2, 3, 4, 5, 6, 7, 8, 9, 10, 11, 12, 13, 14, 15, 16, 17, 18, 19, 20 or more glycine residues. In some embodiments, the amino acid sequence of keratin comprises 3 or more glycine residues. In some embodiments, the amino acid sequence of keratin comprises 5 or more glycine residues. In some embodiments, the amino acid sequence of keratin comprises 7 or more glycine residues. In some embodiments, the amino acid sequence of keratin comprises 10 or more glycine residues.

In some embodiments, at least about 5% of the keratin amino acid sequence comprises glycine. In some embodiments, at least about 6% of the keratin amino acid sequence comprises glycine. In some embodiments, at least about 8% of the keratin amino acid sequence comprises glycine. In some embodiments, at least about 10%, 12%, 15%, 17%, 20%, 22%, 25%, 27%, or 30% of the keratin amino acid sequence comprises glycine.

In some embodiments, the keratin comprises one or more disulfide bonds formed between two or more amino acid residues of keratin. In some embodiments, the keratin comprises one or more hydrogen bonds formed between two or more amino acid residues of keratin.

In some embodiments, the percent composition of keratin is at least about 10%, 15%, 20%, 25%, 30%, 35%, 40%, 45%, 50%, 55%, 60%, 65%, 70%, 75%, 80%, 85%, 90%, or 95% w/w of the total composition. In some embodiments, the percent composition of keratin is at least about 30% w/w of the total composition. In some embodiments, the percent composition of keratin is at least about 40% w/w of the total composition. In some embodiments, the percent composition of keratin is at least about 50% w/w of the total composition.

In some embodiments, the percent composition of keratin is less than about 97%, 95%, 90%, 85%, 80%, 75%, 70%, 65% or 60% w/w of the total composition. In some embodiments, the percent composition of keratin is less than about 97% w/w of the total composition. In some embodiments, the percent composition of keratin is less than about 95% w/w of the total composition. In some embodiments, the percent composition of keratin is less than about 90% w/w of the total composition. In some embodiments, the percent composition of keratin is less than about 87% w/w of the total composition. In some embodiments, the percent composition of keratin is less than about 85% w/w of the total composition. In some embodiments, the percent composition of keratin is less than about 80% w/w of the total composition.

In some embodiments, the keratin composition further comprises melanin. In some embodiments, the percent composition of melanin is at least about 0.01%, 0.02%, 0.03%, 0.04%, 0.5%, 0.06%, 0.07%, 0.08%, 0.09%, 0.10%, 0.20%, 0.50%, 0.70%, 0.8%, 0.9%, 1%, 1.5%, 2%, 2.5%, 3%, 3.5%, 4%, 4.5%, 5%, 5.5%, 6%, 7%, 8%, 9%, or 10% w/w of the total keratin composition. In some embodiments, the percent composition of melanin is at least about 0.5% w/w of the total keratin composition. In some embodiments, the percent composition of melanin is at least about 1% w/w of the total keratin composition. In some embodiments, the percent composition of melanin is at least about 5% w/w of the total keratin composition.

In some embodiments, the percent composition of melanin is less than about 97%, 95%, 90%, 85%, 80%, 75%, 70%, 65%, 60%, 55%, 50%, 45%, 40%, 35%, 30%, 25%, 20%, 15%, or 10% w/w of the total keratin composition. In some embodiments, the percent composition of melanin is less than about 97% w/w of the total keratin composition. In some embodiments, the percent composition of melanin is less than about 95% w/w of the total keratin composition. In some embodiments, the percent composition of melanin is less than about 90% w/w of the total keratin composition. In some embodiments, the percent composition of melanin is less than about 80% w/w of the total keratin composition. In some embodiments, the percent composition of melanin is less than about 70% w/w of the total keratin composition. In some embodiments, the percent composition of melanin is less than about 60% w/w of the total keratin composition. In some embodiments, the percent composition of melanin is less than about 50% w/w of the total keratin composition. In some embodiments, the percent composition of melanin is less than about 40% w/w of the total keratin composition. In some embodiments, the percent composition of melanin is less than about 30% w/w of the total keratin composition.

In some embodiments, the keratin composition further comprises calcium. In some embodiments, the percent composition of calcium is at least about 0.01%, 0.02%, 0.03%, 0.04%, 0.5%, 0.06%, 0.07%, 0.08%, 0.09%, 0.10%, 0.20%, 0.50%, 0.70%, 0.8%, 0.9%, 1%, 1.5%, 2%, 2.5%, 3%, 3.5%, 4%, 4.5%, 5%, 5.5%, 6%, 7%, 8%, 9%, or 10% w/w of the total keratin composition. In some embodiments, the percent composition of calcium is at least about 0.5% w/w of the total keratin composition. In some embodiments, the percent composition of calcium is at least about 1% w/w of the total keratin composition. In some embodiments, the percent composition of calcium is at least about 5% w/w of the total keratin composition.

In some embodiments, the percent composition of calcium is less than about 97%, 95%, 90%, 85%, 80%, 75%, 70%, 65%, 60%, 55%, 50%, 45%, 40%, 35%, 30%, 25%, 20%, 15%, or 10% w/w of the total keratin composition. In some embodiments, the percent composition of calcium is less than about 97% w/w of the total keratin composition. In some embodiments, the percent composition of calcium is less than about 95% w/w of the total keratin composition. In some embodiments, the percent composition of calcium is less than about 90% w/w of the total keratin composition. In some embodiments, the percent composition of calcium is less than about 80% w/w of the total keratin composition. In some embodiments, the percent composition of calcium is less than about 70% w/w of the total keratin composition. In some embodiments, the percent composition of calcium is less than about 60% w/w of the total keratin composition. In some embodiments, the percent composition of calcium is less than about 50% w/w of the total keratin composition. In some embodiments, the percent composition of calcium is less than about 40% w/w of the total keratin composition. In some embodiments, the percent composition of calcium is less than about 30% w/w of the total keratin composition.

In some embodiments, the keratin composition further comprises water. In some embodiments, the percent composition of water is at least about 5%, 10%, 15%, 20%, 25%, 30%, 35%, 40%, 45%, 50%, 55%, 60%, 65%, or 70% w/w of the total keratin composition. In some embodiments, the percent composition of water is at least about 10% w/w of the total keratin composition. In some embodiments, the keratin composition of the water is at least about 20% w/w of the total keratin composition. In some embodiments, the keratin composition of the water is at least about 40% w/w of the total keratin composition.

In some embodiments, the percent composition of water is less than about 95%, 90%, 85%, 80%, 75%, 70%, 65%, 60%, 55%, 50%, 45%, or 40% w/w of the total keratin composition. In some embodiments, the percent composition of water is less than about 90% w/w of the total keratin composition. In some embodiments, the keratin composition of the water is less than about 80% w/w of the total keratin composition. In some embodiments, the keratin composition of the water is less than about 70% w/w of the total keratin composition. In some embodiments, the keratin composition of the water is less than about 60% w/w of the total keratin composition. In some embodiments, the keratin composition of the water is less than about 50% w/w of the total keratin composition.

In some embodiments, keratin forms a two-stranded molecule. In some embodiments, keratin forms one or more intermediate filaments. In some embodiments, the density of the intermediate filaments is at least about 5 mm⁻². In some embodiments, the density of the intermediate filament is at least about 6 mm⁻². In some embodiments, the density of the intermediate filament is at least about 7 mm⁻². In some embodiments, the diameter of the intermediate filament is at least about 50 μm. In some embodiments, the diameter of the intermediate filament is at least about 70 μm. In some embodiments, the diameter of the intermediate filament is at least about 100 μm. In some embodiments, the diameter of the intermediate filament is at least about 200, 300, 400, 500, 600, 700, 800, 900, or 1000 μm.

In some embodiments, the intermediate filaments are embedded in a matrix. In some embodiments, the matrix is an amorphous protein matrix. In some embodiments, the matrix comprises a keratin matrix. In some embodiments, the matrix comprises a non-crystalline keratin matrix.

In some embodiments, the keratin surrounds a core. In some embodiments, the core is a hair-like core. In some embodiments, the core is a non-fibrous core. In some embodiments, the core is solid.

In some embodiments, the keratin composition is in the shape of a horn. In other embodiments, the keratin composition is in the shape of a tusk.

In some embodiments, the keratin composition further comprises cholesterol, taurine, hexosamine, phospholipid, or a combination thereof.

In some embodiments, the keratin composition is a powder. Alternatively, the keratin composition is an aqueous solution. The keratin composition can be a tablet, capsule,

In some embodiments, the keratin composition further comprises one or more excipients. Examples of excipients include, but are not limited to, antiadherents, binders, coatings, disintegrants, fillers, flavours, colours, lubricants, glidants, sorbents, preservatives, and sweeteners.

In some embodiments, the keratin composition comprises an antiadherent. In some instances, antiadherents are used to reduce the adhesion between the powder (granules) and the punch faces and thus prevent sticking to tablet punches. In some embodiments, they are also used to help protect tablets from sticking. In some embodiments, the anti adherent is magnesium stearate.

In some embodiments, the keratin composition comprises a binder. In some instances, binders hold the ingredients in a tablet together. Binders may ensure that tablets and granules can be formed with required mechanical strength, and give volume to low active dose tablets. In some instances, binders are saccharides and their derivatives, protein, and synthetic polymers. Examples of saccharides and their derivatives include, but are not limited to, disaccharides (e.g., sucrose, lactose) and polysaccharides and their derivatives (e.g., starches, cellulose or modified cellulose such as microcrystalline cellulose and cellulose ethers such as hydroxypropyl cellulose (HPC)), and sugar alcohols (e.g., xylitol, sorbitol or maltitol). In some embodiments, the protein binder is gelatin. Examples of synthetic polymers include, but are not limited to, polyvinylpyrrolidone (PVP) and polyethylene glycol (PEG).

In some embodiments, binders are classified according to their application. In some embodiments, the binder is a solution binder. In some embodiments, solution binders are dissolved in a solvent (for example water or alcohol can be used in wet granulation processes). Examples include gelatin, cellulose, cellulose derivatives, polyvinylpyrrolidone, starch, sucrose and polyethylene glycol.

In some embodiments, the binder is a dry binder. In some embodiments, dry binders are added to the powder blend, either after a wet granulation step, or as part of a direct powder compression (DC) formula. Examples include cellulose, methyl cellulose, polyvinylpyrrolidone and polyethylene glycol.

In some embodiments, the keratin composition comprises a coating. In some embodiments, the coating is a tablet coating. The tablet coating may protect tablet ingredients from deterioration by moisture in the air and make large or unpleasant-tasting tablets easier to swallow. In some embodiments, the coated tablet comprises a cellulose ether hydroxypropyl methylcellulose (HPMC) film coating. In some embodiments, coatings comprise synthetic polymers, shellac, corn protein zein or other polysaccharides. In some embodiments, capsules are coated with gelatin.

In some embodiments, the keratin composition comprises a disintegrant. In some embodiments, the disintegrant expands and dissolves when wet causing the tablet to break apart in the digestive tract, releasing the active ingredients for absorption. In some embodiments, the disintegrate ensures that when the tablet is in contact with water, it rapidly breaks down into smaller fragments, facilitating dissolution.

Examples of disintegrants include, but are not limited to, crosslinked polymers and modified starch sodium starch glycolate. In some embodiments, crosslinked polymers comprise crosslinked polyvinylpyrrolidone (crospovidone), crosslinked sodium carboxymethyl cellulose (croscarmellose sodium).

In some embodiments, the keratin composition comprises a filler (e.g., bulking agent, diluent). In some instances, fillers add volume and/or mass to a drug substance, thereby facilitating precise metering and handling thereof in the preparation of dosage forms. Fillers may fill out the size of a tablet or capsule, thereby making it practical to produce and convenient for the consumer to use.

In some embodiments, the filler is inert, compatible with the other components of the formulation, non-hygroscopic, relatively cheap, compactible, and/or tasteless or pleasant tasting. In some embodiments, the filler comprises a plant cellulose (pure plant filler). Alternatively, the filler is a dibasic calcium phosphate. In some instances, the filler comprises vegetable fats and oils. Other examples of fillers include, but are not limited to, lactose, sucrose, glucose, mannitol, sorbitol, calcium carbonate, and magnesium stearate.

In some embodiments, the keratin composition further comprises a flavor. In some instances, flavors are used to mask unpleasant tasting active ingredients and improve the acceptance that the patient will complete a course of medication. Flavorings may be natural (e.g. fruit extract) or artificial.

In some embodiments, the keratin composition further comprises one or more colors. Colors can be added to improve the appearance of the keratin composition.

In some embodiments, the keratin composition further comprises one or more lubricants. Lubricants may prevent ingredients from clumping together and from sticking to the tablet punches or capsule filling machine. Lubricants may also ensure that tablet formation and ejection can occur with low friction between the solid and die wall.

Examples of lubricants include, but are not limited to, common minerals such as talc or silica, and fats (e.g. vegetable stearin, magnesium stearate or stearic acid). In some embodiments, lubricants are agents added in small quantities to tablet and capsule formulations to improve certain processing characteristics.

In some embodiments, lubricants are hydrophilic. In other instances, lubricants are hydrophobic.

In some embodiments, the keratin composition further comprises one or more glints. In some embodiments, glidants are used to promote powder flow by reducing interparticle friction and cohesion. Glidants may be used in combination with lubricants as they have no ability to reduce die wall friction. Examples of glidants include, but are not limited to, fumed silica, talc, and magnesium carbonate.

In some embodiments, the keratin composition further comprises a sorbent. In some embodiments, sorbents are used for tablet/capsule moisture-proofing by limited fluid sorbing (taking up of a liquid or a gas either by adsorption or by absorption) in a dry state. Sorbents may comprise fatty acids, waxes, shellac, plastics, and plant fibers.

In some embodiments, the keratin composition further comprises a preservative. In some embodiments, preservatives include, but are not limited to, antioxidants like vitamin A, vitamin E, vitamin C, retinyl palmitate, and selenium; the amino acids cysteine and methionine; citric acid and sodium citrate; and synthetic preservatives like the parabens: methyl paraben and propyl paraben.

In some embodiments, the keratin composition further comprises a sweetener. Sweeteners may be added to make the keratin composition more palatable, especially in chewable tablets such as antacid or liquids like cough syrup.

### 3D Printing

In some embodiments, methods of manufacturing keratin compositions comprises the use of one or more printers. In some embodiments, the 3D printer is used to manufacture a 3-dimensional keratin composition (e.g., horn, tusk, ornamental or decorative product, handle, knife handle, dagger handle). In some embodiments, the 3D printer is used to manufacture the one or more scaffolds. In some embodiments, the one or more printers are 3D printers. In some embodiments, the one or more printers are materials printers. In some embodiments, the one or more printers are Objet Connex series printers or 3D Systems' ProJet series printers.

3D printing technology has been used for both prototyping and distributed manufacturing in jewelry, footwear, industrial design, architecture, engineering and construction (AEC), automotive, aerospace, dental and medical industries, education, geographic information systems, civil engineering, and many other fields. Generally, 3D printing (e.g., additive manufacturing) refers to a process of making a three-dimensional solid object of virtually any shape from a digital model. In some embodiments, 3D printing is achieved using an additive process, where successive layers of material are laid down in different shapes. In some embodiments, a materials printer performs 3D printing processes using digital technology.

In some embodiments, methods of manufacturing keratin compositions further comprise producing one or more blueprints. In some embodiments, the one or more blueprints are virtual blueprints. In some embodiments, the one or more blueprints are produced from one or more computer software. In some embodiments, the one or more computer software comprise computer aided design (CAD). Alternatively, or additionally, the one or more computer software program comprise animation modeling software.

In some embodiments, the method further comprises slicing the one or more blueprints into digital cross-sections. Slicing the one or more blueprints may be performed virtually. In some embodiments, the digital cross-sections are used as a guideline for printing.

In some embodiments, material or a binding material is deposited on the build bed or platform until material/binder layering is complete and the final 3D model has been printed. In some embodiments, the virtual model (e.g., blueprint) and the physical model (e.g., printed product) are almost identical. In some embodiments, the material or binding material comprise one or more cells. In some embodiments, the material or binding material comprise keratin. In some embodiments, the material or binding material comprise a keratin powder.

In some embodiments, a standard data interface between CAD software and the machines is the STL file format. In some embodiments, an STL file approximates the shape of a part or assembly using triangular facets Smaller facets may produce a higher quality surface. In some embodiments, PLY is a scanner generated input file format. VRML (or WRL) files may be used as input for 3D printing technologies that are able to print in full color.

In some embodiments, printing a keratin product comprises (a) reading a design from a computer file; and (b) laying down successive layers of liquid, powder, paper or sheet material to build a model from a series of cross sections. In some embodiments, these layers, which correspond to the virtual cross sections from the CAD model, are joined together or automatically fused to create the final shape. In some embodiments, reading a design from a computer file comprises the use of one or more computers or computer processors. In some embodiments, the computer file is an .stl file. In some embodiments, the liquid, powder, paper or sheet material comprises one or more cells (e.g., stem cells, iPSC, keratinocyte, terminally differentiated cell). In some embodiments, the liquid, powder, paper or sheet material comprises keratin. In some embodiments, the liquid, powder, paper or sheet material comprises calcium. In some embodiments, the liquid, powder, paper or sheet material comprises melanin.

In some embodiments, layer thickness is at least about 16 micrometers. In some embodiments, layer thickness is at least about 20, 25, 30, 35, 40, 45, 50, 55, 60, 65, 70, 75, 80, 85, 90, or 95 micrometers. In some embodiments, layer thickness is about 100 micrometers (0.1 mm). In some embodiments, the layer thickness is less than about 1000, 900, 800, 700, 600, 500, 400, 300, or 200 micrometers.

In some embodiments, X-Y resolution of the 3D printer is comparable to that of laser printers. In some embodiments, the particles (e.g., 3D dots) are at least about 10, 15, 20, 25, 30, 35, 40, 45, or 50 micrometers. In some embodiments, the particles (e.g., 3D dots) are less than about 1000, 900, 800, 700, 600, 500, 400, 300, 250, 200, 150, or 100 micrometers in diameter. In some embodiments, the particles (e.g., 3D dots) are around 50 to 100 micrometers (0.05-0.1 mm) in diameter.

In some embodiments, the method further comprises finishing a keratin product. Finishing a keratin product may comprise printing a slightly oversized version of the desired object (e.g., rhino horn, elephant tusk, decorative handle) in standard resolution. In some embodiments finishing the keratin product may further comprise removing material from the slightly oversized version with a higher-resolution subtractive process.

In some embodiments, 3D printing techniques use multiple materials (e.g., cells, keratin, melanin, calcium) in the course of constructing parts. In some embodiments, 3D printing techniques print in multiple colors and color combinations. In some embodiments, printing in multiple colors and color combinations occurs simultaneously. In some embodiments, printing in multiple colors and color combinations occurs sequentially. In some embodiments, 3D printing utilizes supports when building. In some instances, supports are removable or dissolvable upon completion of the print. Supports may be used to support overhanging features during construction.

Further disclosed herein are systems for manufacturing keratin products. In some embodiments, the system comprises (a) a memory device for receiving one or more blueprints; (b) a computer software program for modifying the one or more blueprints; (c) an ink cartridge comprising one or more rhinoceros cells or one or keratin compositions produced from one or more rhinoceros cells; and (d) a printer for printing a 3D keratin product with the ink cartridge. The one or more rhinoceros cells may be a stem cell or iPSC. The one or more rhinoceros cells may be a rhino fibroblast. The one or more rhinoceros cells may be a somatic cell. The one or more rhinoceros cells may be a rhino keratinocyte. The one or more rhinoceros cells may be a terminally differentiated cell.

In some embodiments, the system further comprises one or more additional memory devices for receiving instructions for printing the 3D keratin product. In some embodiments, the system further comprises one or more additional memory devices for receiving instructions for finishing the 3D keratin product. In some embodiments, the system further comprises one or more processors for finishing the 3D keratin product. In some embodiments, the system further comprises one or more software programs for digitally slicing the one or more blueprints. In some embodiments, the ink cartridge further comprises melanin. In some embodiments, the ink cartridge further comprises calcium. In some embodiments, the system further comprises one or more additional ink cartridges. The one or more additional ink cartridges may comprise melanin, calcium, water or a combination thereof.

### Uses of Keratin Compositions

The keratin compositions disclosed herein may be used to produce 3-dimensional keratin product. In some embodiments, producing a 3-dimensional (3D) keratin product comprises printing a 3-dimensional keratin product with ink comprising a keratin composition disclosed herein. In some embodiments, the method further comprises providing one or more blueprints of the 3D keratin product. In some embodiments, the method further comprises finishing the 3D keratin product.

In some embodiments, producing a 3D keratin product comprises printing a 3D keratin product with ink comprising one or more cells. The one or more cells may be stem cells. Alternatively, the one or more cells are iPSC. In some embodiments, the one or more cells are keratinocytes. In some embodiments, the one or more cells are terminally differentiated cells. In some embodiments, the method further comprises providing one or more blueprints of the 3D keratin product. In some embodiments, the method further comprises finishing the 3D keratin product. In some embodiments, the method further comprises inducing the one or more cells to produce keratin. In some embodiments, the method further comprises contacting the one or more cells with one or more solutions.

Alternatively, producing a 3D keratin product comprises seeding the keratin composition onto a scaffold. In some embodiments, producing a 3D keratin product comprises seeding one or more cells comprising one or more keratin genes onto a scaffold. In some embodiments, the one or more cells are stem cells, iPSC, keratinocytes, and/or terminally differentiated cells. In some embodiments, the one or more cells are transfected with one or more keratin genes. In some embodiments, the one or more cells are transfected with one or more melanin genes.

3D keratin products may refer to In some embodiments, the keratin products are synthetically-derived rhinoceros horns. The synthetically-derived rhinoceros horn may appear identical to a naturally produced rhinoceros horn. The synthetically-derived rhinoceros horn may appear at least 50% identical to a naturally produced rhinoceros horn. The synthetically-derived rhinoceros horn may appear at least 60%, 65%, 70%, 75%, 80%, 85%, 90%, 95% or more identical to a naturally produced rhinoceros horn. The composition of the synthetically-derived rhinoceros horn may be at least about 50% identical to the composition of a naturally produced rhinoceros horn. The composition of the synthetically-derived rhinoceros horn may be at least about 60%, 65%, 70%, 75%, 80%, 85%, 90%, 95% or more identical to the composition of a naturally produced rhinoceros horn. The percent composition of keratin of the synthetically-derived rhinoceros horn may be at least about 50% identical to the percent composition of keratin of a naturally produced rhinoceros horn. The percent composition of keratin of the synthetically-derived rhinoceros horn may be at least about 60%, 65%, 70%, 75%, 80%, 85%, 90%, 95% or more identical to the percent composition of keratin of a naturally produced rhinoceros horn. The percent composition of melanin of the synthetically-derived rhinoceros horn may be at least about 50% identical to the percent composition of melanin of a naturally produced rhinoceros horn. The percent composition of melanin of the synthetically-derived rhinoceros horn may be at least about 60%, 65%, 70%, 75%, 80%, 85%, 90%, 95% or more identical to the percent composition of melanin of a naturally produced rhinoceros horn. The percent composition of calcium of the synthetically-derived rhinoceros horn may be at least about 50% identical to the percent composition of calcium of a naturally produced rhinoceros horn. The percent composition of keratin of the synthetically-derived rhinoceros horn may be at least about 60%, 65%, 70%, 75%, 80%, 85%, 90%, 95% or more identical to the percent composition of calcium of a naturally produced rhinoceros horn.

FIG. 2 shows the shape of rhinoceros horns. In some embodiments, synthetically-derived rhinoceros horns comprise a similar shape as the rhinoceros horns depicted in FIG. 2. In some embodiments, the synthetically-derived rhinoceros horns comprises similar coloring as naturally occurring rhinoceros horns. In some embodiments, the synthetically-derived rhinoceros horns comprises similar striations as naturally occurring rhinoceros horns.

The term “naturally produced rhinoceros horn” refers to a rhinoceros horn produced from a rhinoceros. The term “synthetically-derived rhinoceros horn” refers to a rhinoceros horn that is not produced from a rhinoceros. Synthetically-derived rhinoceros horn may be produced from one or more cells. Alternatively, or additionally, the synthetically-derived rhinoceros horn is produced by 3D printing.

In other embodiments, the keratin products are synthetically-derived elephant tusks. The synthetically-derived elephant tusk may appear identical to a naturally produced elephant tusk. The synthetically-derived elephant tusk may appear at least 50% identical to a naturally produced elephant tusk. The synthetically-derived elephant tusk may appear at least 60%, 65%, 70%, 75%, 80%, 85%, 90%, 95% or more identical to a naturally produced elephant tusk. The composition of the synthetically-derived elephant tusk may be at least about 50% identical to the composition of a naturally produced elephant tusk. The composition of the synthetically-derived elephant tusk may be at least about 60%, 65%, 70%, 75%, 80%, 85%, 90%, 95% or more identical to the composition of a naturally produced elephant tusk. The percent composition of keratin of the synthetically-derived elephant tusk may be at least about 50% identical to the percent composition of keratin of a naturally produced elephant tusk. The percent composition of keratin of the synthetically-derived elephant tusk may be at least about 60%, 65%, 70%, 75%, 80%, 85%, 90%, 95% or more identical to the percent composition of keratin of a naturally produced elephant tusk. The percent composition of melanin of the synthetically-derived elephant tusk may be at least about 50% identical to the percent composition of melanin of a naturally produced elephant tusk. The percent composition of melanin of the synthetically-derived elephant tusk may be at least about 60%, 65%, 70%, 75%, 80%, 85%, 90%, 95% or more identical to the percent composition of melanin of a naturally produced elephant tusk. The percent composition of calcium of the synthetically-derived elephant tusk may be at least about 50% identical to the percent composition of calcium of a naturally produced elephant tusk. The percent composition of keratin of the synthetically-derived elephant tusk may be at least about 60%, 65%, 70%, 75%, 80%, 85%, 90%, 95% or more identical to the percent composition of calcium of a naturally produced elephant tusk.

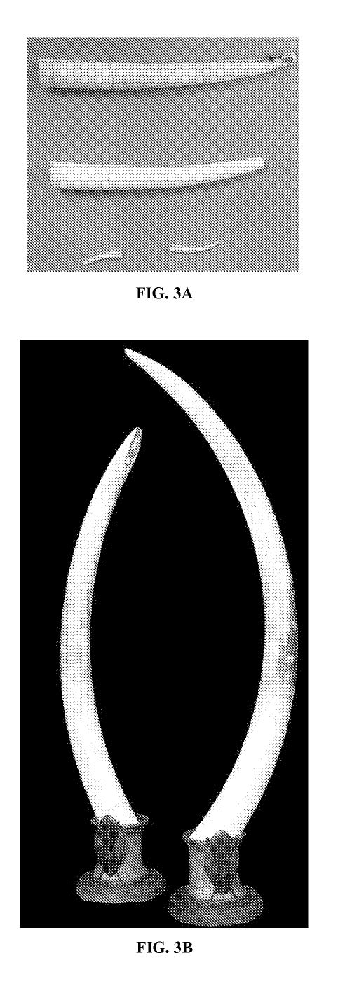

FIG. 3A shows the shape of elephant tusks. In some embodiments, synthetically-derived elephant tusks comprise a similar shape as the elephant tusks depicted in FIG. 3A. In some embodiments, the synthetically-derived elephant tusks comprises similar coloring as naturally occurring elephant tusks. In some embodiments, the synthetically-derived elephant tusks comprises similar striations as naturally occurring elephant tusks.

The term “naturally produced elephant tusk” refers to an elephant tusk produced from an elephant. The term “synthetically-derived elephant tusk” refers to a elephant tusk that is not produced from an elephant. Synthetically-derived elephant tusk may be produced from one or more cells. Alternatively, or additionally, the synthetically-derived elephant tusk is produced by 3D printing.

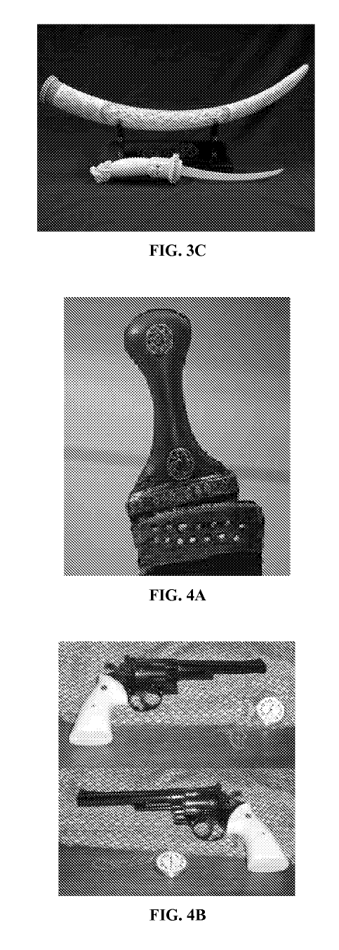

In some embodiments, the keratin products are handles. In some embodiments, the handles are knife handles or dagger handles. In some embodiments, the keratin compositions are used for the handles of curved daggers. In some embodiments, the curved daggers are called jambiya. FIG. 4A depicts an exemplary handle of a curved dagger.

In some embodiments, the handles are gun handles. In some embodiments, the keratin product comprises a gun handle. FIG. 4B depicts an exemplary gun handle comprising keratin.

In some embodiments, the handles are handles for doors, drawers, or furniture pulls. In other embodiments, the handles are for cups. In other embodiments, the handles are for utensils (e.g., forks, knives, spoons).

### EXAMPLES

### Example 1

### Synthesis of a Rhinoceros Keratinocyte

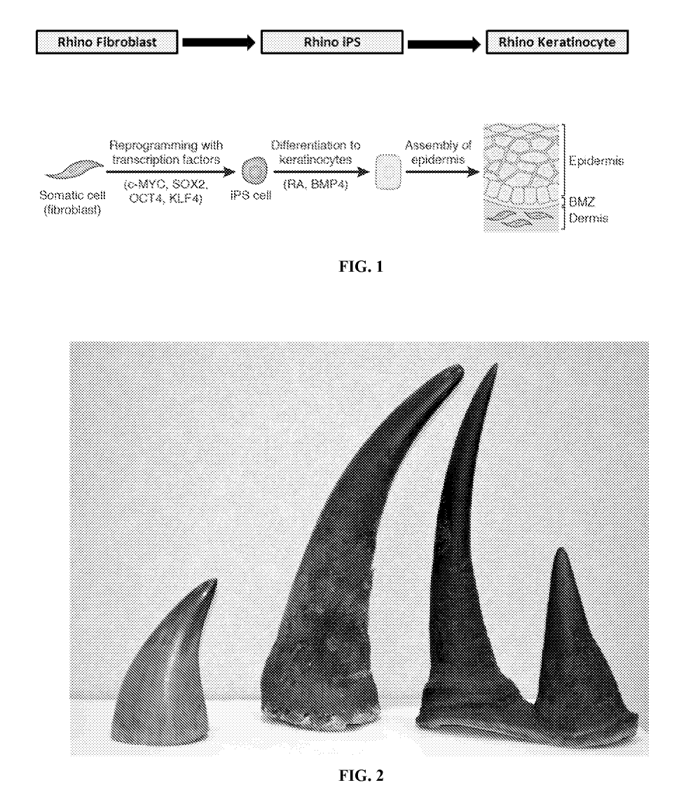

FIG. 1 depicts a general schematic for producing a rhinoceros keratinocyte. Generally, a somatic cell is reprogrammed with one or more transcription factors to produce an induced pluripotent stem cell (iPSC). The iPSC is then differentiated to produce a keratinocyte.

In this example, a rhinoceros keratinocyte is synthesized from a somatic cell. A rhino fibrobast is transfected with retroviral vectors comprising sequences for the reprogramming factors POU5F1 (OCT4), SOX2, KLF4 and Myc to produce a rhino induced pluripotent stem cell (iPSC). The fibroblasts are screened for morphology and karyotic normality. The integration of the viral vectors is confirmed by PCR.

Rhino iPSCs are selected based on morphology, karyotype, immunostaining and increased expression of stem cell markers (OCT4, Sox2, NANOG), silencing of the viral transgenes, demethylation of stem cell gene promoters (NANOG), teratoma formation in vivo and/or contribution to all three germ layers. The rhino iPSC cells are differentiated into keratinocytes by treatment with retinoic acid (RA) and/or bone morphogenic protein 4 (BMP4).

Keratinocyte formation is confirmed by the presence of keratin-14.

TABLE 1
Keratin-Nucleotide Sequences

| NAME | SEQ ID NO | SEQUENCE |
|---|---|---|
| Keratin 1 | 1 | agaggagtgtttagctccttcccttactctaccttgctcctacttttctctaagt |
| | | caacatgagtcgacagtttagttccaggtctgggtaccgaagtggagggggcttc |
| | | agctctggctctgctgggatcatcaactaccagcgcaggaccaccagcagctcca |
| | | cacgccgcagtggaggaggtggtgggagattttcaagctgtggtggtggtggtgg |
| | | tagctttggtgctggtggtggatttggaagtcggagtcttgttaaccttggtggc |
| | | agtaaaagcatctccataagtgtggctagaggaggtggacgtggtagtggctttg |
| | | gtggtggttatggtggtggtggctttggtggtggtggctttggtggtggtggctt |
| | | tggtggaggtggcattgggggtggtggctttggtggttttggcagtggtggtggt |
| | | ggttttggtggaggtggctttgggggtggtggatatgggggtggttatggtcctg |
| | | tctgccctcctggtggcatacaagaagtcactatcaaccagagccttcttcagcc |
| | | cctcaatgtggagattgaccctgagatccaaaaggtgaagtctcgagaaagggag |
| | | caaatcaagtcactcaacaaccaatttgcctccttcattgacaaggtgaggttcc |
| | | tggagcagcagaaccaggtactgcaaacaaaatgggagctgctgcagcaggtaga |
| | | tacctccactagaacccataatttagagccctactttgagtcattcatcaacaat |
| | | ctccgaaggagagtggaccaactgaagagtgatcaatctcggttggattcggaac |
| | | tgaagaacatgcaggacatggtggaggattaccggaacaagtatgaggatgaaat |
| | | caacaagcggacaaatgcagagaatgaatttgtgaccatcaagaaggatgtggat |
| | | ggtgcttatatgaccaaggtggaccttcaggccaaacttgacaacttgcagcagg |
| | | aaattgatttccttacagcactctaccaagcagagttgtctcagatgcagactca |
| | | aatcagtgaaactaatgtcatcctctctatggacaacaaccgcagtctcgacctg |
| | | gacagcatcattgctgaggtcaaggcccagtacgaggatatagcccagaagagca |
| | | aagctgaggccgagtccttgtaccagagcaagtatgaagagctgcagatcactgc |
| | | tggcagacatggggatagtgtgagaaattcaaagatagaaatttctgagctgaat |
| | | cgtgtgatccagagacttagatctgaaatcgacaatgtcaagaagcagatctcca |
| | | acttgcagcagtccatcagtgatgcagagcagcgtggcgagaatgccctcaagga |
| | | tgccaagaacaagctgaatgacctggaggatgccctgcagcaggccaaggaagac |
| | | ctggcccgcctgctgcgcgactaccaggagctgatgaacaccaagctggccctgg |
| | | atctggagattgccacctacaggaccctcctggagggagaagaaagcaggatgtc |
| | | tggagaatgtgccccgaacgtgagtgtgtctgtgagcacaagccacaccaccatc |
| | | agtggaggtggcagccgaggaggtggcggcggtggctacggctctggaggtagca |
| | | gctatggctccggaggtggtagctatggttctggaggtggcggcggcggcggccg |
| | | tggcagctatggctccggaggtagcagctacggctccggaggtggcagctatggc |
| | | tctggaggtggcggcggcggccatggcagctacggctccggaagcagcagtgggg |
| | | gctacagaggtggctctggaggcggcggcggcggcagctctggcggccggggctc |
| | | tggcggcgggagctctggaggctccataggaggccggggatccagctctgggggt |
| | | gtcaagtcctctggtggcagttccagcgtgaagtttgtttctaccacttattccg |
| | | gagtaaccagataaagagatgccctctgtttcattagctctagttctcccccagc |
| | | atcactaacaaatatgcttggcaagaccgaggtcgatttgtcccagccttaccgg |
| | | agaaaagagctatggttagttacactagctcatcctattcccccagctctttctt |
| | | ttctgctgtttcccaatgaagttttcagatcagtggcaatctcagtcccctggct |
| | | atgaccctgctttgttctttccctgagaaacagttcagcagtgaccaccacccac |
| | | atgacatttcaaagcacctccttaagccagccagagtaggaccagttagacccag |
| | | ggtgtggacagctccttagcatcttatctctgtgctgttttggttttgtacataa |
| | | ggtgtaagcaagttgtttttcttttgtggagaggtcaaaactccccataccagat |
| | | tgctgcaataaactgcatagaaattc |
| Keratin5 | 2 | tcgacagctctctcgcccagcccagactggaagggataaaaagggggcatcaccg |
| | | acctgggtaacagagccaccactgcgtcctgctgagctctgactctccagcacct |
| | | cccaacccactagtgcctggactcagctccaccaggaacaagccaccatgtctcg |
| | | ccagtcaagtgtgtccaccggagcgggggcagtcgtagcttcagcaccgcctctg |
| | | ccatcaccccgtctgtctcccgcaccagcttcacctccgtgtcccggtccggggg |
| | | tggcggtggtggtggcttcggcagggtcagccagcgggtgcagtggagtgggtgg |
| | | ctatggcagccggagcctctacaacctggggggctccaagaggatatccatcagc |
| | | actagtggtggcagcacaggaaccggtaggtgctggtgctggaggcggctatggc |
| | | tttggaggtggtgccggtagtggatttggtttcggcggtggagctggtggtggct |
| | | ttgggctcggtggcggagctggctttggaggtggcttcggtggccctggctttcc |
| | | tgtctgccctcctggaggtatccaagaggtcactgtcaaccagagtctcctgact |
| | | cccctcaacctgcaaatcgaccccagcatccagagggtgaggaccgaggagcgcg |
| | | agcagatcaagaccctcaacaataagtttgcctccttcatcgacaaggtgcggtt |
| | | cctggagcagcagaacaaggttctggacaccaagtggaccctgctgcaggagcag |
| | | ggcaccaagactgtgaggcagaacctggagccgttgttcgagcagtacatcaaca |
| | | acctcaggaggcagctggacagcatcgtgggggaacggggccgcctggactcaga |
| | | gctgagaaacatgcaggacctggtggaagacttcaagaacaagtatgaggatgaa |
| | | atcaacaagcgtaccactgctgagaatgagtttgtgatgctgaagaaggatgtag |
| | | atgctgcctacatgaacaaggtggagctggaggccaaggttgatgcactgatgga |
| | | tgagattaacttcatgaagatgttctttgatgcggagctgtcccagatgcagacg |
| | | catgtctctgacacctcagtggtcctctccatggacaacaaccgcaacctggacc |
| | | tggatagcatcatcgctgaggtcaaggcccagtatgaggagattgccaaccgcag |
| | | ccggacagaagccgagtcctggtatcagaccaagtatgaggagctgcagcagaca |
| | | gctggccggcatggcgatgacctccgcaacaccaagcatgagatctctgagatga |
| | | accggatgatccagaggctgagagccgagattgacaatgtcaagaaacagtgcgc |
| | | caatctgcagaacgccattgcggatgccgagcagcgtggggagctggccctcaag |
| | | gatgccaggaacaagctggccgagctggaggaggccctgcagaaggccaagcagg |
| | | acatggcccggctgctgcgtgagtaccaggagctcatgaacaccaagctggccct |
| | | ggacgtggagatcgccacttaccgcaagctgctggagggcgaggaatgcagactc |
| | | agtggagaaggagttggaccagtcaacatctctgttgtcacaagcagtgtttcct |
| | | ctggatatggcagtggcagtggctatggcggtggcctcggtggaggtcttggcgg |
| | | cggcctcggtggaggtcttgccggaggtagcagtggaagctactactccagcagc |
| | | agtgggggtgtcggcctaggtggtgggctcagtgtggggggctctggcttcagtg |
| | | caagcagtggccgagggctgggggtgggctttggcagtggcgggggtagcagctc |
| | | cagcgtcaaatttgtctccaccacctcctcctcccggaagagcttcaagagctaa |
| | | gaacctgctgcaagtcactgccttccaagtgcagcaacccagcccatggagattg |
| | | cctcttctaggcagttgctcaagccatgattatccttttctggagagtagtctag |
| | | accaagccaattgcagaaccacattctttggttcccaggagagccccattcccag |
| | | cccctggtctcccgtgccgcagttctatattctgcttcaaatcagccttcaggtt |
| | | tcccacagcatggcccctgctgacacgagaacccaaagttttcccaaatctaaat |
| | | catcaaaacagaatccccaccccaatcccaaattttgattggttctaactacctc |
| | | cagaatgtgttcaataaaatgcttttataatataaaaaaaaaaaaaaaaaaa |
| Keratin10 | 3 | cactccctgggctaaacagcatcaccatgtctgttcgatacagctcaagcaagca |
| | | ctactcttcctcccgcagtggaggaggaggaggaggaggaggatgtggaggagga |
| | | ggaggagtgtcatccctaagaatttctagcagcaaaggctcccttggtggaggat |
| | | ttagctcaggggggttcagtggtggctcttttagccgtgggagctctggtggggg |
| | | ctgctttgggggctcatcaggtggctatggaggattaggaggttttggtggaggt |
| | | agctttcgtggaagctatggaagtagcagctttggtgggagttatggaggcagct |
| | | ttggagggggcagtttcggaggtggcagctttggtgggggcagctttggtggagg |
| | | cggctttggtggaggcggctttggaggaggctttggtggtggatttggaggagat |
| | | ggtggccttctctctggaaatgaaaaagtaaccatgcagaatctgaatgaccgcc |
| | | tggcttcctacttggacaaagttcgggctctggaagaatcaaactatgagctgga |
| | | aggcaaaatcaaggagtggtatgaaaagcatggcaactcacatcagggggagcct |
| | | cgtgactacagcaaatactacaaaaccatcgatgaccttaaaaatcagattctca |
| | | acctaacaactgataatgccaacatcctgcttcagatcgacaatgccaggctggc |
| | | agctgatgacttcaggctgaagtatgagaatgaggtagctctgcgccagagcgtg |
| | | gaggctgacatcaacggcctgcgtagggtgctggatgagctgaccctgaccaagg |
| | | ctgacctggagatgcaaattgagagcctgactgaagagctggcctatctgaagaa |
| | | gaaccacgaggaggaaatgaaagaccttcgaaatgtgtccactggtgatgtgaat |
| | | gtggaaatgaatgctgccccgggtgttgatctgactcaacttctgaataacatga |
| | | gaagccaatatgaacaacttgctgaacaaaaccgcaaagatgctgaagcctggtt |
| | | caatgaaaagagcaaggaactgactacagaaattgataataacattgaacagata |
| | | tccagctataaatctgagattactgaattgagacgtaatgtacaagctctggaga |
| | | tagaactacagtcccaactggccttgaaacaatccctggaagcctccttggcaga |
| | | aacagaaggtcgctactgtgtgcagctctcacagattcaggcccagatatccgct |
| | | ctggaagaacagttgcaacagattcgagctgaaaccgagtgccagaatactgaat |
| | | accaacaactcctggatattaagatccgactggagaatgaaattcaaacctaccg |
| | | cagcctgctagaaggagagggaagttccggaggcggcggacgcggcggcggaagt |
| | | ttcggcggcggctacggcggcggaagctccggcggcggaagctccggcggcggcc |
| | | acggcggcggccacggcggcagttccggcggcggctacggaggcggaagctccgg |
| | | cggcggaagctccggcggcggctacgggggcggaagctccagcggcggccacggc |
| | | ggcagttccagcggcggctacggtggtggcagttccggcggcggcggcggcggct |
| | | acgggggcggcagctccggcggcggcagcagctccggcggcggatacggcggcgg |
| | | cagctccagcggaggccacaagtcctcctcttccgggtccgtgggcgagtcttca |
| | | tctaagggaccaagatactaacaaaaccagagtaatcaagacaattattgaagag |
| | | gtggcgcccgacggtagagttctttcatctatggttgaatcagaaaccaagaaac |
| | | actactattaaactgcatcaagaggaaagagtctcccttcacacagaccattatt |
| | | tacagatgcatggaaaacaaagtctccaagaaaacacttctgtcttgatggtcta |
| | | tggaaatagaccttgaaaataaggtgtctacaaggtgattgtggtttctgtattt |
| | | cttcttttcactttaccagaaagtgttctttaatggaaagaaaaacaactttctg |
| | | ttctcatttactaatgaatttcaataaactttcttactgatgcaaactaaaaaaa |
| | | aaaaaaaaaaaaaaaa |
| Keratin14 | 4 | acccgagcaccttctcttcactcagccaactgctcgctcgctcacctccctcctc |
| | | tgcaccatgaccacctgcagccgccagttcacctcctccagctccatgaagggct |
| | | cctgcggcatcgggggcggcatcgggggcggctccagccgcatctcctccgtcct |
| | | ggccggagggtcctgccgcgcccccagcacctacgggggcggcctgtctgtctca |
| | | tcctcccgcttctcctctgggggagcctacgggctggggggcggctatggcggtg |
| | | gcttcagcagcagcagcagcagctttggtagtggctttgggggaggatatggtgg |
| | | tggccttggtgctggcttgggtggtggctttggtggtggctttgctggtggtgat |
| | | gggcttctggtgggcagtgagaaggtgaccatgcagaacctcaatgaccgcctgg |
| | | cctcctacctggacaaggtgcgtgctctggaggaggccaacgccgacctggaagt |
| | | gaagatccgtgactggtaccagaggcagcggcctgctgagatcaaagactacagt |
| | | ccctacttcaagaccattgaggacctgaggaacaagattctcacagccacagtgg |
| | | acaatgccaatgtccttctgcagattgacaatgcccgtctggccgcggatgactt |
| | | ccgcaccaagtatgagacagagttgaacctgcgcatgagtgtggaagccgacatc |
| | | aatggcctgcgcagggtgctggacgaactgaccctggccagagctgacctggaga |
| | | tgcagattgagagcctgaaggaggagctggcctacctgaagaagaaccacgagga |
| | | ggagatgaatgccctgagaggccaggtgggtggagatgtcaatgtggagatggac |
| | | gctgcacctggcgtggacctgagccgcattctgaacgagatgcgtgaccagtatg |
| | | agaagatggcagagaagaaccgcaaggatgccgaggaatggttcttcaccaagac |
| | | agaggagctgaaccgcgaggtggccaccaacagcgagctggtgcagagcggcaag |
| | | agcgagatctcggagctccggcgcaccatgcagaacctggagattgagctgcagt |
| | | cccagctcagcatgaaagcatccctggagaacagcctggaggagaccaaaggtcg |
| | | ctactgcatgcagctggcccagatccaggagatgattggcagcgtggaggagcag |
| | | ctggcccagctccgctgcgagatggagcagcagaaccaggagtacaagatcctgc |
| | | tggacgtgaagacgcggctggagcaggagatcgccacctaccgccgcctgctgga |
| | | gggcgaggacgcccacctctcctcctcccagttctcctctggatcgcagtcatcc |
| | | agagatgtgacctcctccagccgccaaatccgcaccaaggtcatggatgtgcacg |
| | | atggcaaggtggtgtccacccacgagcaggtccttcgcaccaagaactgaggctg |
| | | cccagccccgctcaggcctaggaggccccccgtgtggacacagatcccactggaa |
| | | gatcccctctcctgcccaagcacttcacagctggaccctgcttcaccctcacccc |
| | | ctcctggcaatcaatacagcttcattatctgagttgcataaaaaaaaaaaaaaaa |
| | | aaa |

TABLE 2
Keratin-Amino Acid sequences

| NAME | SEQ ID NO | SEQUENCE |
|---|---|---|
| Keratin1 | 5 | MSRQFSSRSGYRSGGGFSSGSAGIINYQRRTTSSSTRRSGGGGGRFSSCGGGGGSFGA |
| | | GGGFGSRSLVNLGGSKSISISVARGGGRGSGFGGGYGGGGFGGGGFGGGGFGGGGIGG |
| | | GGFGGFGSGGGGFGGGGFGGGGYGGGYGPVCPPGGIQEVTINQSLLQPLNVEIDPEIQ |
| | | KVKSREREQIKSLNNQFASFIDKVRFLEQQNQVLQTKWELLQQVDTSTRTHNLEPYFE |
| | | SFINNLRRRVDQLKSDQSRLDSELKNMQDMVEDYRNKYEDEINKRTNAENEFVTIKKD |
| | | VDGAYMTKVDLQAKLDNLQQEIDFLTALYQAELSQMQTQISETNVILSMDNNRSLDLD |
| | | SIIAEVKAQYEDIAQKSKAEAESLYQSKYEELQITAGRHGDSVRNSKIEISELNRVIQ |
| | | RLRSEIDNVKKQISNLQQSISDAEQRGENALKDAKNKLNDLEDALQQAKEDLARLLRD |
| | | YQELMNTKLALDLEIATYRTLLEGEESRMSGECAPNVSVSVSTSHTTISGGGSRGGGG |
| | | GGYGSGGSSYGSGGGSYGSGGGGGGGRGSYGSGGSSYGSGGGSYGSGGGGGGHGSYGS |
| | | GSSSGGYRGGSGGGGGGSSGGRGSGGGSSGGSIGGRGSSSGGVKSSGGSSSVKFVSTT |
| | | YSGVTR |
| Keratin5 | 6 | MSRQSSVSFRSGGSRSFSTASAITPSVSRTSFTSVSRSGGGGGGGFGRVSLAGACGVG |
| | | GYGSRSLYNLGGSKRISISTSGGSFRNRFGAGAGGGYGFGGGAGSGFGFGGGAGGGFG |
| | | LGGGAGFGGGFGGPGFPVCPPGGIQEVTVNQSLLTPLNLQIDPSIQRVRTEEREQIKT |
| | | LNNKFASFIDKVRFLEQQNKVLDTKWTLLQEQGTKTVRQNLEPLFEQYINNLRRQLDS |
| | | IVGERGRLDSELRNMQDLVEDFKNKYEDEINKRTTAENEFVMLKKDVDAAYMNKVELE |
| | | AKVDALMDEINFMKMFFDAELSQMQTHVSDTSVVLSMDNNRNLDLDSIIAEVKAQYEE |
| | | IANRSRTEAESWYQTKYEELQQTAGRHGDDLRNTKHEISEMNRMIQRLRAEIDNVKKQ |
| | | CANLQNAIADAEQRGELALKDARNKLAELEEALQKAKQDMARLLREYQELMNTKLALD |
| | | VEIATYRKLLEGEECRLSGEGVGPVNISVVTSSVSSGYGSGSGYGGGLGGGLGGGLGG |
| | | GLAGGSSGSYYSSSSGGVGLGGGLSVGGSGFSASSGRGLGVGFGSGGGSSSSVKFVST |
| | | TSSSRKSFKS |
| Keratin10 | 7 | MSVRYSSSKHYSSSRSGGGGGGGGCGGGGGVSSLRISSSKGSLGGGFSSGGFSGGSFS |
| | | RGSSGGGCFGGSSGGYGGLGGFGGGSFRGSYGSSSFGGSYGGSFGGGSFGGGSFGGGS |
| | | FGGGGFGGGGFGGGFGGGFGGDGGLLSGNEKVTMQNLNDRLASYLDKVRALEESNYEL |
| | | EGKIKEWYEKHGNSHQGEPRDYSKYYKTIDDLKNQILNLTTDNANILLQIDNARLAAD |
| | | DFRLKYENEVALRQSVEADINGLRRVLDELTLTKADLEMQIESLTEELAYLKKNHEEE |
| | | MKDLRNVSTGDVNVEMNAAPGVDLTQLLNNMRSQYEQLAEQNRKDAEAWFNEKSKELT |
| | | TEIDNNIEQISSYKSEITELRRNVQALEIELQSQLALKQSLEASLAETEGRYCVQLSQ |
| | | IQAQISALEEQLQQIRAETECQNTEYQQLLDIKIRLENEIQTYRSLLEGEGSSGGGGR |
| | | GGGSFGGGYGGGSSGGGSSGGGHGGGHGGSSGGGYGGGSSGGGSSGGGYGGGSSSGGH |
| | | GGSSSGGYGGGSSGGGGGGYGGGSSGGGSSSGGGYGGGSSSGGHKSSSSGSVGESSSK |
| | | GPRY |
| Keratin14 | 8 | MTTCSRQFTSSSSMKGSCGIGGGIGGGSSRISSVLAGGSCRAPSTYGGGLSVSSSRFS |
| | | SGGAYGLGGGYGGGFSSSSSSFGSGFGGGYGGGLGAGLGGGFGGGFAGGDGLLVGSEK |
| | | VTMQNLNDRLASYLDKVRALEEANADLEVKIRDWYQRQRPAEIKDYSPYFKTIEDLRN |
| | | KILTATVDNANVLLQIDNARLAADDFRTKYETELNLRMSVEADINGLRRVLDELTLAR |
| | | ADLEMQIESLKEELAYLKKNHEEEMNALRGQVGGDVNVEMDAAPGVDLSRILNEMRDQ |
| | | YEKMAEKNRKDAEEWFFTKTEELNREVATNSELVQSGKSEISELRRTMQNLEIELQSQ |
| | | LSMKASLENSLEETKGRYCMQLAQIQEMIGSVEEQLAQLRCEMEQQNQEYKILLDVKT |
| | | RLEQEIATYRRLLEGEDAHLSSSQFSSGSQSSRDVTSSSRQIRTKVMDVHDGKVVSTH |
| | | EQVLRTKN |

1. A method for producing a rhinoceros keratinocyte comprising contacting one or more embryoid bodies with one or more solutions to produce a rhinoceros keratinocyte, wherein:
a. the one or more embryoid bodies are formed from a rhinoceros induced pluripotent stem cell (rhino iPSC); and
b. the one or more solutions comprise one or more nutrients, one or more growth factors, or a combination thereof.

2. The method of claim 1, wherein the one or more solutions comprise embryonic stem cell (ESC) medium.

3. The method of claim 2, wherein the ESC medium does not comprise leukemia inhibitory factor (LIF).

4. The method of claim 1, wherein the one or more solutions comprise a first solution and a second solution.

5. (canceled)

6. (canceled)

7. The method of claim 1, wherein the one or more nutrients are selected from the group comprising retinoic acid, retinol, amino acids, purines, pyrimidines, vitamins, glucose, and inorganic ions.

8. The method of claim 1, wherein the one or more nutrients comprise retinoic acid.

9. The method of claim 1, wherein the one or more growth factors are selected from the group comprising bone-morphogenetic protein-4 (BMP-4), BMP-2, BMP-6, BMP-7, epidermal growth factor (EGF), fibroblast growth factor 2 (FGF-2), granulocyte colony stimulating factor (G-CSF), granulocyte/macrophage colony stimulating factor (GM-CSF), insulin like growth factor I (IGF-I), and vascular endothelial growth factor (VEGF).

10. The method of claim 1, wherein the one or more growth factors comprise bone-morphogenetic protein-4 (BMP-4).

11. The method of claim 1, further comprising culturing the rhinoceros keratinocyte on a coated cell culture surface.

12. (canceled)

13. The method of claim 11, wherein the coated cell culture surface is coated with collagen, fibronectin, poly-D-lysine, gelatin, laminin, hydrogel, extracellular matrix (ECM) or a combination thereof.

14. The method of claim 13, wherein collagen is collagen I, collagen IV, or a combination thereof.

15. The method of claim 11, wherein the coated cell culture surface is coated with collagen IV.

16. The method of claim 1, further comprising culturing the one or more embryoid bodies or the rhinoceros keratinocytes in embryonic stem cell (ESC) medium.

17. The method of claim 16, wherein the ESC medium does not comprise leukemia inhibitory factor (LIF).

18. The method of claim 1, further comprising culturing the rhinoceros keratinocytes in keratinocyte serum-free medium (KSFM).

19. The method of claim 1, further comprising differentiating the rhinoceros keratinocyte to produce a differentiated keratinocyte.

20. The method of claim 19, wherein differentiating the rhinoceros keratinocyte comprises contacting the rhinoceros keratinocyte with a culture medium comprising calcium (Ca²⁺).

21. -25. (canceled)

26. The method of claim 1, wherein the rhino iPSC expresses POU5F1, SOX2, NANOG, or a combination thereof.

27. The method of claim 1, wherein the rhinoceros keratinocyte expresses Krt14, Krt10, or a combination thereof.

28. The method of claim 19, wherein the differentiated keratinocyte expresses Krt1, loricrin, or a combination thereof.

29. -139. (canceled)
